(19) United States
(12) Patent Application Publication
(10) Pub. No.: US 2010/0222353 A1
(43) Pub. Date: Sep. 2, 2010

(54) AMINOPHTHALAZINE DERIVATIVE COMPOUNDS

(75) Inventor: John Michael Humphrey, Mystic, CT (US)

Correspondence Address: PFIZER INC;Mary J Hosley, 150 EAST 42ND STREET, MS: 150/02/E112, NEW YORK, NY, 10017-5612, US

(73) Assignee: PFIZER PRODUCTS INC., Groton, CT (US)

(21) Appl. No.: 12/161,718

(22) PCT Filed: Jan. 22, 2007
(86) PCT No.: PCT/IB2007/000197

§ 371 Date: Jul. 22, 2008

Related U.S. Application Data
(60) Provisional application No. 60/763,019, filed on Jan. 27, 2006.

(51) Int. Cl. A61K 31/502; C07D 403/04; C07D 401/04; C07D 401/14; C07D 413/14; A61P 25/22; A61P 25/30; A61P 25/00; A61P 25/28; A61P 25/16; A61P 25/24; A61P 25/18

(52) U.S. Cl. 514/248; 544/237

(57) ABSTRACT

The invention pertains to new aminophthalazine compounds that serve as effective phosphodiesterase (PDE) inhibitors. The invention also relates to compounds that are selective inhibitors of PDE-10. The invention further relates to intermediates for preparation of such compounds; pharmaceutical compositions comprising such compounds; and the use of such compounds in methods for treating certain central nervous system (CNS) or other disorders. The invention relates also to methods for treating neurodegenerative and psychiatric disorders, for example psychosis and disorders comprising deficient cognition as a symptom.

### FIELD OF THE INVENTION

The invention pertains to new aminophthalazine compounds that serve as effective phosphodiesterase (PDE) inhibitors. The invention also relates to compounds that are selective inhibitors of PDE-10. The invention further relates to intermediates for preparation of such compounds; pharmaceutical compositions comprising such compounds; and the use of such compounds in methods for treating certain central nervous system (CNS) or other disorders. The invention relates also to methods for treating neurodegenerative and psychiatric disorders, for example psychosis and disorders comprising deficient cognition as a symptom.

### BACKGROUND OF INVENTION

Phosphodiesterases (PDEs) are a class of intracellular enzymes involved in the hydrolysis of the nucleotides cyclic adenosine monophosphate (cAMP) and cyclic guanosine monophosphates (cGMP) into their respective nucleotide monophosphates. The cyclic nucleotides cAMP and cGMP are synthesized by adenylyl and guanylyl cyclases, respectively, and serve as secondary messengers in several cellular pathways.

The cAMP and cGMP function as intracellular second messengers regulating a vast array of intracellular processes particularly in neurons of the central nervous system. In neurons, this includes the activation of cAMP and cGMP-dependent kinases and subsequent phosphorylation of proteins involved in acute regulation of synaptic transmission as well as in neuronal differentiation and survival. The complexity of cyclic nucleotide signaling is indicated by the molecular diversity of the enzymes involved in the synthesis and degradation of cAMP and cGMP. There are at least ten families of adenylyl cyclases, two of guanylyl cyclases, and eleven of phosphodiesterases. Furthermore, different types of neurons are known to express multiple isozymes of each of these classes, and there is good evidence for compartmentalization and specificity of function for different isozymes within a given neuron.

A principal mechanism for regulating cyclic nucleotide signaling is by phosphodiesterase-catalyzed cyclic nucleotide catabolism. There are 11 known families of PDEs encoded by 21 different genes. Each gene typically yields multiple splice variants that further contribute to the isozyme diversity. The PDE families are distinguished functionally based on cyclic nucleotide substrate specificity, mechanism(s) of regulation, and sensitivity to inhibitors. Furthermore, PDEs are differentially expressed throughout the organism, including in the central nervous system. As a result of these distinct enzymatic activities and localization, different PDEs' isozymes can serve distinct physiological functions. Furthermore, compounds that can selectively inhibit distinct PDE families or isozymes may offer particular therapeutic effects, fewer side effects, or both.

PDE10 is identified as a unique family based on primary amino acid sequence and distinct enzymatic activity. Homology screening of EST databases revealed mouse PDE10A as the first member of the PDE10 family of PDEs (Fujishige et al., J. Biol. Chem. 274:18438-18445, 1999; Loughney, K. et al., Gene 234:109-117, 1999). The murine homologue has also been cloned (Soderling, S. et al., Proc. Natl. Acad. Sci. USA 96:7071-7076, 1999) and N-terminal splice variants of both the rat and human genes have been identified (Kotera, J. et al., Biochem. Biophys. Res. Comm. 261:551-557, 1999; Fujishige, K. et al., Eur. J. Biochem. 266:1118-1127, 1999). There is a high degree of homology across species. The mouse PDE10A1 is a 779 amino acid protein that hydrolyzes both cAMP and cGMP to AMP and GMP, respectively. The affinity of PDE10 for cAMP (Km=0.05 μM) is higher than for cGMP (Km=3 μM). However, the approximately 5-fold greater Vmax for cGMP over cAMP has lead to the suggestion that PDE10 is a unique cAMP-inhibited cGMPase (Fujishige et al., J. Biol. Chem. 274:18438-18445, 1999).

PDE10 also is uniquely localized in mammals relative to other PDE families. mRNA for PDE10 is highly expressed only in testis and brain (Fujishige, K. et al., Eur J Biochem. 266:1118-1127, 1999; Soderling, S. et al., Proc. Natl. Acad. Sci. 96:7071-7076, 1999; Loughney, K. et al., Gene 234:109-117, 1999). These initial studies indicated that within the brain PDE10 expression is highest in the striatum (caudate and putamen), n. accumbens, and olfactory tubercle. More recently, a detailed analysis has been made of the expression pattern in rodent brain of PDE10 mRNA (Seeger, T. F. et al., Abst. Soc. Neurosci. 26:345.10, 2000) and PDE10 protein (Menniti, F. S., Stick, C. A., Seeger, T. F., and Ryan, A. M., Immunohistochemical localization of PDE10 in the rat brain. William Harvey Research Conference ‘Phosphodiesterase in Health and Disease’, Porto, Portugal, Dec. 5-7, 2001).

United States Patent Application Publication No. 2003/0032579 discloses a method for treating certain neurologic and psychiatric disorders with the selective PDE10 inhibitor papaverine. In particular, the method relates to psychotic disorders such as schizophrenia, delusional disorders and drug-induced psychosis; to anxiety disorders such as panic and obsessive-compulsive disorder; and to movement disorders including Parkinson's disease and Huntington's disease.

In their role as second messengers in intracellular signaling events, cAMP and cGMP affect a wide array of processes including neurotransmission and enzyme activation. Intracellular levels of these chemicals are largely maintained by two classes of enzymes in response to other cellular stimuli. The first of these enzymes, the adenylyl and guanylyl cyclases, catalyze the formation of cAMP and cGMP thereby raising their concentrations and activating certain signaling events. The second enzyme class, the phosphodiesterases (PDE's), catalyzes the degradation of cAMP and cGMP, which results in termination of the signal.

Signal enhancement via elevation of cyclic nucleotide concentration can be induced through employment of PDE inhibitors. The present invention describes the use of such PDE inhibitors as therapies for the prevention or treatment of diseases linked to abnormal cell signaling processes, and relates to compounds described below.

### SUMMARY OF THE INVENTION

The invention relates to compounds having the following formula, denoted herein as formula I:

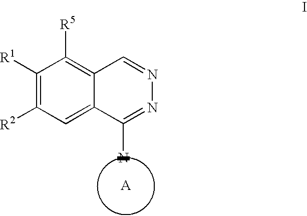

or a pharmaceutically acceptable salt thereof,

wherein ring A is a 5 or 6 membered heterocyclic ring substituted by at least one R⁶ and at least one R⁷;

wherein R¹, R² and R⁶ are each independently H, halogen, —CN, —COOH, —COOR³, —CONR³R⁴, —COR²⁰, —NR³R⁴, —NHCOR²⁰, —OH, (C₆-C₁₀)aryl, (5-10) membered heteroaryl, (C₁-C₆)alkyl, (C₁-C₆)haloalkyl (C₂-C₆)alkenyl, (C₂-C₆)alkynyl, (C₁-C₆)alkoxy, (C₂-C₆)alkenyloxy or (C₃-C₈) cycloalkyl; or, when R¹, R² and R⁵ are independently (C₁-C₆)alkoxy, (C₂-C₆)alkenyloxy, (C₁-C₆)alkyl, (C₂-C₆)alkenyl or (C₂-C₆)alkynyl, R¹ and R² or R¹ and R⁵ may optionally be connected to form a 5 to 8 membered ring;

wherein R³ and R⁴ are each independently H, (C₁-C₆)alkyl (5-10 membered)heteroaryl or (C₆-C₁₀)aryl, wherein said heteroaryl or aryl may be optionally substituted with one or more (C₁-C₆)alkyl or halo groups;

wherein each R⁶ is independently H, halogen, —COOR³, —CONR³R⁴, —COR²⁰, —NR³R⁴, —OH, (C₁-C₆)hydroxyalkyl —HNCOOR³, —CN, —HNCONHR⁴, (C₁-C₆)alkyl, (C₁-C₆)alkoxy, (C₆-C₁₀)aryl, —O—(C₁-C₆)alkylene-(5-8 membered)heteroaryl, —O—(C₁-C₆)alkylene-(C₆-C₁₀)aryl, —(C₁-C₆)alkylene-O-(5-8 membered)heteroaryl, —(C₁-C₆)alkylene-O—(C₆-C₁₀)aryl or

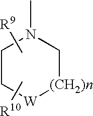

wherein n is 0 or 1;

W is carbon, oxygen or NR⁸, wherein R⁸ is hydrogen or (C₁-C₆)alkyl, and when W is carbon, it may be optionally substituted by halogen, —CN, —COOH, —COOR³, —CONR³R⁴, —COR²⁰, —NR³R⁴, —NHCOR²⁰, —OH, (C₆-C₁₀)aryl, (5-10) membered heteroaryl, (C₁-C₆)alkyl, (C₁-C₆)haloalkyl (C₂-C₆)alkenyl, (C₂-C₆)alkynyl, (C₁-C₆)alkyloxy, (C₂-C₆)alkenyloxy or (C₃-C₈)cycloalkyl; and wherein said alkyl, aryl or heteroaryl of R⁶ may be optionally substituted by (C₁-C₈)alkyl, (C₃-C₈)cycloalkyl, (C₁-C₈)alkoxy, halogen, —OH, and halo(C₁-C₈)alkyl;

wherein R⁹ and R¹⁰ are independently hydrogen or (C₁-C₈)alkyl;

or R⁹ and R¹⁰ may optionally combine to form a cyclic ring;

wherein each R⁷ is independently R¹¹, —R¹⁸—R¹¹ or —OR¹¹;

wherein R¹¹ is hydrogen, phenyl, naphthyl, or a 5- to 6-membered heteroaryl ring, optionally fused to a benzo group or heteroaryl ring, containing from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈)alkyl, (C₃-C₈)cycloalkyl, (C₁-C₈)alkoxy, halogen, —CN, —OH, (C₆-C₁₀)aryl, (5-10) membered heteroaryl, halo(C₁-C₈)alkyl, (C₁-C₈)hydroxyalkyl, (C₁-C₈)alkoxy-(C₁-C₈)alkyl, (C₃-C₈)hydroxycycloalkyl, (C₃-C₈)cycloalkoxy, (C₁-C₈)alkoxy-(C₃-C₈)cycloalkyl, (C₃-C₈)heterocycloalkyl, (C₃-C₆)hydroxyheterocycloalkyl, and (C₁-C₈)alkoxy-heterocycloalkyl, wherein each cycloalkyl or heterocycloalkyl moiety may be independently substituted with from one to three halogens, (C₁-C₆)alkyl or benzyl groups; or

when R¹¹ is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR¹⁴R¹⁵ or —(C₀-C₆)alkylene-NR¹⁴R¹⁵, wherein R¹⁴ and R¹⁵ are each independently selected from hydrogen, (C₁-C₈)alkyl and benzyl, or R¹⁴ and R¹⁵ together with the nitrogen to which they are attached form a (5-7) membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; (c) —(CH₂)ᵥNCOR¹⁶R¹⁷, wherein v is zero, one, two or three and —COR¹⁶ and R¹⁷ taken together with the nitrogen to which they are attached may form a 4- to 6-membered lactam ring; or

when R¹¹ is heteroaryl, it may be optionally fused to ring A and optionally substituted with —NR¹²R¹³;

wherein R¹², R¹³, R¹⁶ and R¹⁷ are each independently hydrogen, (C₁-C₆)alkyl, and (C₆-C₁₀)aryl;

wherein R¹⁸ is (C₁-C₃) alkylene or —N(R₁₉)—; wherein said alkylene may be optionally substituted by (C₁-C₈)alkyl, (C₃-C₈)cycloalkyl, (C₁-C₈)alkoxy, halogen, —OH, or halo(C₁-C₈)alkyl;

R¹⁹ is hydrogen or (C₁-C₆)alkyl; and

wherein each R²⁰ is independently (C₁-C₈)alkyl, (C₃-C₈)cycloalkyl (C₁-C₈)alkoxy, halo(C₁-C₈)alkyl, (C₁-C₈)hydroxyalkyl, (C₁-C₈)alkoxy-(C₁-C₈)alkyl, (C₃-C₈)hydroxycycloalkyl, (C₃-C₈)cycloalkoxy, (C₁-C₈)alkoxy-(C₃-C₈)cycloalkyl, (C₃-C₈)heterocycloalkyl, (C₆-C₁₀)aryl or (5-10) membered heteroaryl.

### DETAILED DESCRIPTION OF THE INVENTION

Compounds of the Formula I may have optical centers and therefore may occur in different enantiomeric and diastereomeric configurations. The present invention includes all enantiomers, diastereomers, and other stereoisomers of such compounds of the Formula I, as well as racemic compounds and racemic mixtures and other mixtures of stereoisomers thereof.

Pharmaceutically acceptable salts of the compounds of Formula I include the acid addition and base salts thereof.

Suitable acid addition salts are formed from acids that form non-toxic salts. Examples include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable base salts are formed from bases that form non-toxic salts. Examples include, but are not limited to, the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of Formula I may be prepared by one or more of three methods:

(i) by reacting the compound of Formula I with the desired acid or base;

(ii) by removing an acid- or base-labile protecting group from a suitable precursor of the compound of Formula I or by ring-opening a suitable cyclic precursor, for example, a lactone or lactam, using the desired acid or base; or

(iii) by converting one salt of the compound of Formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of Formula I include references to salts, solvates, multi-component complexes and liquid crystals thereof and to solvates, multi-component complexes and liquid crystals of salts thereof.

The compounds of the invention include compounds of Formula I as hereinbefore defined, including all polymorphs and crystal habits thereof, prodrugs and isomers thereof (including optical, geometric and tautomeric isomers) as hereinafter defined and isotopically-labeled compounds of Formula I.

In one embodiment of the present invention, ring A is piperidine or pyrrolidine.

In another embodiment of the present invention, R¹ and R² are each independently (C₁-C₄)alkoxy or methoxy.

In another embodiment of the present invention, R⁷ is R¹¹ and R¹¹ is phenyl optionally substituted by (C₁-C₆)alkoxy, (C₁-C₅)alkyl, —CN, —OH, phenyl or (C₁-C₆)alkoxy substituted with 1 to 3 halogens

In another embodiment of the present invention, R₇ is —OR¹¹ and R¹¹ is naphthyl or naphthyl substituted by (C₁-C₆)alkoxy or (C₁-C₆)alkyl.

In another embodiment of the present invention, R⁷ is —OR¹¹ and R¹¹ is 5 or 6 membered heteroaryl.

In another embodiment of the present invention, wherein R⁶ is (C₁-C₆)alkyloxy or —OH.

In another embodiment of the present invention, R⁶ is —NR³R⁴, and R³ and R⁴ are each independently (C₁-C₃)alkyl.

In another embodiment of the present invention, wherein R¹ and R² are each independently (C₁-C₆)alkoxy, R⁷ is R¹¹ and R¹¹ is phenyl or substituted phenyl and R⁶ is (C₁-C₆)alkoxy or —OH.

In another embodiment, R⁶ and R⁷ can not both be hydrogen.

As indicated, so-called ‘prodrugs’ of the compounds of Formula I are also within the scope of the invention. Thus certain derivatives of compounds of Formula I which may have little or no pharmacological activity themselves can, when administered into or onto the body, be converted into compounds of Formula I having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of Formula I with certain moieties known to those skilled in the art as ‘pro-moieties’ as described, for example, in Design of Prodrugs by H. Bundgaard (Elsevier, 1985).

Some examples of prodrugs in accordance with the invention include, but are not limited to,

(i) where the compound of Formula I contains a carboxylic acid functionality (—COOH), an ester thereof, for example, a compound wherein the hydrogen of the carboxylic acid functionality of the compound of Formula (I) is replaced by (C₁-C₈)alkyl;

(ii) where the compound of Formula I contains an alcohol functionality (—OH), an ether thereof, for example, a compound wherein the hydrogen of the alcohol functionality of the compound of Formula I is replaced by (C₁-C₆)alkanoyloxymethyl; and

(iii) where the compound of Formula I contains a primary or secondary amino functionality (—NH₂ or —NHR where R≠H), an amide thereof, for example, a compound wherein, as the case may be, one or both hydrogens of the amino functionality of the compound of Formula I is/are replaced by (C₁-C₁₀)alkanoyl.

Further examples of replacement groups in accordance with the foregoing examples and examples of other prodrug types may be found in the aforementioned references.

Moreover, certain compounds of Formula I may themselves act as prodrugs of other compounds of Formula I.

Also included within the scope of the invention are metabolites of compounds of Formula I, that is, compounds formed in vivo upon administration of the drug. Some examples of metabolites in accordance with the invention include, but are not limited to,

(i) where the compound of Formula I contains a methyl group, an hydroxymethyl derivative thereof (—CH₃->—CH₂OH):

(ii) where the compound of Formula I contains an alkoxy group, an hydroxy derivative thereof (—OR->—OH);

(iii) where the compound of Formula I contains a tertiary amino group, a secondary amino derivative thereof (—NR¹R²->—NHR¹ or —NHR²);

(iv) where the compound of Formula I contains a secondary amino group, a primary derivative thereof (—NHR¹->—NH₂);

(v) where the compound of Formula I contains a phenyl moiety, a phenol derivative thereof (-Ph->-PhOH); and

(vi) where the compound of Formula I contains an amide group, a carboxylic acid derivative thereof (—CONH₂->COOH).

Compounds of Formula I containing one or more asymmetric carbon atoms can exist as two or more stereoisomers. Where a compound of Formula I contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of Formula I containing, for example, an imino, keto, or oxime group, or so-called valence tautomerism in compounds that contain an aromatic moiety. It follows that a single compound may exhibit more than one type of isomerism.

Included within the scope of the present invention are all stereoisomers, geometric isomers and tautomeric forms of the compounds of Formula I, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of Formula I wherein one or more atoms are replaced by atoms having the same atomic number, but an atomic mass or mass number different from the atomic mass or mass number which predominates in nature.

Examples of isotopes suitable for inclusion in the compounds of the invention include, but are not limited to, isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula I, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula I can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

This invention also pertains to a pharmaceutical composition for treatment of certain psychotic disorders and conditions such as schizophrenia, delusional disorders and drug induced psychosis; to anxiety disorders such as panic and obsessive-compulsive disorder; and to movement disorders including Parkinson's disease and Huntington's disease, comprising an amount of a compound of formula I effective in inhibiting PDE 10.

In another embodiment, this invention relates to a pharmaceutical composition for treating psychotic disorders and condition such as schizophrenia, delusional disorders and drug induced psychosis; anxiety disorders such as panic and obsessive-compulsive disorder; and movement disorders including Parkinson's disease and Huntington's disease, comprising an amount of a compound of formula I effective in treating said disorder or condition.

Examples of psychotic disorders that can be treated according to the present invention include, but are not limited to, schizophrenia, for example of the paranoid, disorganized, catatonic, undifferentiated, or residual type; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type.

Examples of movement disorders that can be treated according to the present invention include but are not limited to Huntington's disease and dyskinesia associated with dopamine agonist therapy, Parkinson's disease, restless leg syndrome, and essential tremor.

Other disorders that can be treated according to the present invention are obsessive/compulsive disorders, Tourette's syndrome and other tic disorders.

In another embodiment, this invention relates to a method for treating an anxiety disorder or condition in a mammal which method comprises administering to said mammal an amount of a compound of formula I effective in inhibiting PDE 10.

This invention also provides a method for treating an anxiety disorder or condition in a mammal which method comprises administering to said mammal an amount of a compound of formula I effective in treating said disorder or condition.

Examples of anxiety disorders that can be treated according to the present invention include, but are not limited to, panic disorder; agoraphobia; a specific phobia; social phobia; obsessive-compulsive disorder; post-traumatic stress disorder; acute stress disorder; and generalized anxiety disorder.

This invention further provides a method of treating a drug addiction, for example an alcohol, amphetamine, cocaine, or opiate addiction, in a mammal, including a human, which method comprises administering to said mammal an amount of a compound of formula I effective in treating drug addiction.

This invention also provides a method of treating a drug addiction, for example an alcohol, amphetamine, cocaine, or opiate addiction, in a mammal, including a human, which method comprises administering to said mammal an amount of a compound of formula I effective in inhibiting PDE10.

A “drug addiction”, as used herein, means an abnormal desire for a drug and is generally characterized by motivational disturbances such a compulsion to take the desired drug and episodes of intense drug craving.

This invention further provides a method of treating a disorder comprising as a symptom a deficiency in attention and/or cognition in a mammal, including a human, which method comprises administering to said mammal an amount of a compound of formula I effective in treating said disorder.

This invention also provides a method of treating a disorder or condition comprising as a symptom a deficiency in attention and/or cognition in a mammal, including a human, which method comprises administering to said mammal an amount of a compound of formula I effective in inhibiting PDE10.

This invention also provides a method of treating a disorder or condition comprising as a symptom a deficiency in attention and/or cognition in a mammal, including a human, which method comprises administering to said mammal an amount of a compound of formula I effective in treating said disorder or condition.

The phrase “deficiency in attention and/or cognition” as used herein in “disorder comprising as a symptom a deficiency in attention and/or cognition” refers to a subnormal functioning in one or more cognitive aspects such as memory, intellect, or learning and logic ability, in a particular individual relative to other individuals within the same general age population. “Deficiency in attention and/or cognition” also refers to a reduction in any particular individual's functioning in one or more cognitive aspects, for example as occurs in age-related cognitive decline.

Examples of disorders that comprise as a symptom a deficiency in attention and/or cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; and age-related cognitive decline.

This invention also provides a method of treating a mood disorder or mood episode in a mammal, including a human, comprising administering to said mammal an amount of a compound of formula I effective in treating said disorder or episode.

This invention also provides a method of treating a mood disorder or mood episode in a mammal, including a human, comprising administering to said mammal an amount of a compound of formula I effective in inhibiting PDE10.

Examples of mood disorders and mood episodes that can be treated according to the present invention include, but are not limited to, major depressive episode of the mild, moderate or severe type, a manic or mixed mood episode, a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder such as delusional disorder or schizophrenia; a bipolar disorder, for example bipolar I disorder, bipolar II disorder, and cyclothymic disorder.

This invention further provides a method of treating a neurodegenerative disorder or condition in a mammal, including a human, which method comprises administering to said mammal an amount of a compound of formula I effective in treating said disorder or condition.

This invention further provides a method of treating a neurodegenerative disorder or condition in a mammal, including a human, which method comprises administering to said mammal an amount of a compound of formula I effective in inhibiting PDE10.

As used herein, and unless otherwise indicated, a “neurodegenerative disorder or condition” refers to a disorder or condition that is caused by the dysfunction and/or death of neurons in the central nervous system. The treatment of these disorders and conditions can be facilitated by administration of an agent which prevents the dysfunction or death of neurons at risk in these disorders or conditions and/or enhances the function of damaged or healthy neurons in such a way as to compensate for the loss of function caused by the dysfunction or death of at-risk neurons. The term “neurotrophic agent” as used herein refers to a substance or agent that has some or all of these properties.

Examples of neurodegenerative disorders and conditions that can be treated according to the present invention include, but are not limited to, Parkinson's disease; Huntington's disease; dementia, for example Alzheimer's disease, multi-infarct dementia, AIDS-related dementia, and Fronto temperal Dementia; neurodegeneration associated with cerebral trauma; neurodegeneration associated with stroke, neurodegeneration associated with cerebral infarct; hypoglycemia-induced neurodegeneration; neurodegeneration associated with epileptic seizure; neurodegeneration associated with neurotoxin poisoning; and multi-system atrophy.

In one embodiment of the present invention, the neurodegenerative disorder or condition comprises neurodegeneration of striatel medium spiny neurons in a mammal, including a human.

In a further embodiment of the present invention, the neurodegenerative disorder or condition is Huntington's disease.

In another embodiment, this invention provides a pharmaceutical composition for treating psychotic disorders, delusional disorders and drug induced psychosis; anxiety disorders, movement disorders, mood disorders, neurodegenerative disorders and drug addiction, comprising an amount of a compound of formula I effective in treating said disorder or condition.

In another embodiment, this invention provides a method of treating a disorder selected from psychotic disorders, delusional disorders and drug induced psychosis; anxiety disorders, movement disorders, mood disorders, and neurodegenerative disorders, which method comprises administering an amount of a compound of claim 1 effective in treating said disorder.

In another embodiment, this invention provides a method of treating the disorders above, where the disorders are selected from the group consisting of: dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; age-related cognitive decline, major depressive episode of the mild, moderate or severe type; a manic or mixed mood episode; a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder comprising a delusional disorder or schizophrenia; a bipolar disorder comprising bipolar I disorder, bipolar II disorder, cyclothymic disorder, Parkinson's disease; Huntington's disease; dementia, Alzheimer's disease, multi-infarct dementia, AIDS-related dementia, Fronto temperal Dementia; neurodegeneration associated with cerebral trauma; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; multi-system atrophy, paranoid, disorganized, catatonic, undifferentiated or residual type; schizophreniform disorder; schizoaffective disorder of the delusional type or the depressive type; delusional disorder; substance-induced psychotic disorder, psychosis induced by alcohol, amphetamine, cannabis, cocaine, hallucinogens, inhalants, opioids, or phencyclidine; personality disorder of the paranoid type; and personality disorder of the schizoid type.

The term “aryl”, as used herein, unless otherwise indicated, includes an organic radical derived from a univalent aromatic hydrocarbon and includes but is not limited to, phenyl, naphthyl and indenyl.

The term “alkyl”, as used herein, unless otherwise indicated, includes saturated monovalent hydrocarbon radicals having straight or branched moieties. Examples of alkyl groups include, but are not limited to, methyl, ethyl, propyl, isopropyl, and t-butyl.

The term “alkenyl”, as used herein, unless otherwise indicated, includes alkyl moieties having at least one carbon-carbon double bond wherein alkyl is as defined above. Examples of alkenyl include, but are not limited to, ethenyl and propenyl.

The term “alkynyl”, as used herein, unless otherwise indicated, includes alkyl moieties having at least one carbon-carbon triple bond wherein alkyl is as defined above. Examples of alkynyl groups include, but are not limited to, ethynyl and 2-propynyl.

The term “cycloalkyl”, as used herein, unless otherwise indicated, includes alkyl groups comprising non-aromatic saturated cyclic alkyl moieties wherein alkyl is as defined above. Examples of cycloalkyl include, but are not limited to, cyclopropyl, cyclopropylethyl, cyclopropylmethyl, cyclobutyl, cyclopentyl, cyclohexyl, and cycloheptyl.

Unless otherwise indicated, as used herein, the terms “heterocyclic” and “heterocycloalkyl” refer to non-aromatic cyclic groups containing one or more heteroatoms, preferably from one to four heteroatoms, each selected from O, S and N. “Heterobicycloalkyl” groups are non-aromatic two-ringed cyclic groups, wherein said rings share one or two atoms, and wherein at least one of the rings contains a heteroatom (O, S, or N). Heterobicycloalkyl groups for purposes of the present invention, and unless otherwise indicated, include spiro groups and fused ring groups. “Heterotricycloalkyl” groups are non-aromatic three-ringed cyclic groups, wherein said rings are fused to one another or form a spiro group (in other words, at least two of said rings share one or two atoms and the third ring shares one or two atoms with at least one of said two rings). The heterotricycloalkyl groups of the compounds of the present invention can include one or more O, S and/or N heteroatoms. In one embodiment, each ring in the heterobicycloalkyl or heterotricycloalkyl contains up to four heteroatoms (i.e. from zero to four heteroatoms, provided that at least one ring contains at least one heteroatom). The heterocycloalkyl, heterobicycloalkyl and heterotricycloalkyl groups of the present invention can also include ring systems substituted with one or more oxo moieties. The heterocyclic groups, including the heterobicyclic and heterotricyclic groups, may comprise double or triple bonds, e.g. heterocycloalkenyl, heterobicycloalkenyl, and heterotricycloalkenyl. Examples of non-aromatic heterocyclic groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

“Heteroaryl”, as used herein, refers to aromatic groups containing one or more heteroatoms (O, S, or N), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

“Halogen” and “halo”, as used herein, includes chloro, bromo, fluoro and iodo.

“Haloalkyl” as used herein, includes alkyl groups where one or more of the hydrogen atoms are substituted by halogens. Examples of haloalkyl include, but are not limited to —CH₂F, —CHCl₂, —CF₃ and —CH₂CF₃.

Unless otherwise indicated, the term “one or more” substituents, or “at least one” substituent as used herein, refers to from one to the maximum number of substituents possible based on the number of available bonding sites.

“Neurotoxin poisoning” refers to poisoning caused by a neurotoxin. A neurotoxin is any chemical or substance that can cause neural death and thus neurological damage. An example of a neurotoxin is alcohol, which, when abused by a pregnant female, can result in alcohol poisoning and neurological damage known as Fetal Alcohol Syndrome in a newborn. Other examples of neurotoxins include, but are not limited to, kainic acid, domoic acid, and acromelic acid; certain pesticides, such as DDT; certain insecticides, such as organophosphates; volatile organic solvents such as hexacarbons (e.g. toluene); heavy metals (e.g. lead, mercury, arsenic, and phosphorous); aluminum; certain chemicals used as weapons, such as Agent Orange and Nerve Gas; and neurotoxic antineoplastic agents.

As used herein, the term “selective PDE10 inhibitor” refers to a substance, for example an organic molecule that effectively inhibits an enzyme from the PDE10 family to a greater extent than enzymes from the PDE 1-9 families or PDE11 family. In one embodiment, a selective PDE10 inhibitor is a substance, for example an organic molecule, having an IC₅₀ for inhibition of PDE10 that is less than or about one-half the IC₅₀ that the substance has for inhibition of any other PDE enzyme. In other words, the substance inhibits PDE10 activity to the same degree at a concentration of about one-tenth or less than the concentration required for any other PDE enzyme.

In general, a substance is considered to effectively inhibit PDE10 activity if it has an IC₅₀ of less than or about 10 μM, preferably less than or about 0.1 μM.

A “selective PDE10 inhibitor” can be identified, for example, by comparing the ability of a substance to inhibit PDE10 activity to its ability to inhibit PDE enzymes from the other PDE families. For example, a substance may be assayed for its ability to inhibit PDE10 activity, as well as PDE1, PDE2, PDE3, PDE4, PDE5, PDE6, PDE7, PDE8, PDE9, PDE11, including subtypes.

The term “treating”, as in “a method of treating a disorder”, refers to reversing, alleviating, or inhibiting the progress of the disorder to which such term applies, or one or more symptoms of the disorder. As used herein, the term also encompasses, depending on the condition of the patient, preventing the disorder, including preventing onset of the disorder or of any symptoms associated therewith, as well as reducing the severity of the disorder or any of its symptoms prior to onset. “Treating” as used herein refers also to preventing a recurrence of a disorder.

For example, “treating schizophrenia, or schizophreniform or schizoaffective disorder” as used herein also encompasses treating one or more symptoms (positive, negative, and other associated features) of said disorders, for example treating, delusions and/or hallucination associated therewith. Other examples of symptoms of schizophrenia and schizophreniform and schizoaffective disorders include disorganized speech, affective flattening, alogia, anhedonia, inappropriate affect, dysphoric mood (in the form of, for example, depression, anxiety or anger), and some indications of cognitive dysfunction.

In another embodiment the present invention relates to a process for preparing a compound of formula I

comprising reacting a compound of formula III

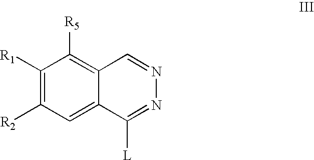

wherein R₁, R₂ and R₅ defined earlier above;

and L is a suitable leaving group; with a compound of formula II

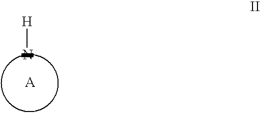

Examples of leaving groups include, but are not limited to chlorine, bromine, iodine, p-toluenesulfonate, C₁-C₆alkylsulfate and C₁-C₆alkanesulfonate, particularly trifluoromethanesulfonate

In a preferred embodiment, the leaving group L is chlorine.

Suitable methods for producing the compounds of the present invention may be found in U.S. Pat. No. 4,370,328, GB 2,000,136 and U.S. Ser. Nos. 11/257,179 and 11/178,104 herein incorporated by reference in their entirety.

The compound of the invention may be administered either alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed thereby can then be readily administered in a variety of dosage forms such as tablets, powders, lozenges, liquid preparations, syrups, injectable solutions and the like. These pharmaceutical compositions can optionally contain additional ingredients such as flavorings, binders, excipients and the like. Thus, the compound of the invention may be formulated for oral, buccal, intranasal, parenteral (e.g. intravenous, intramuscular or subcutaneous), transdermal (e.g. patch) or rectal administration, or in a form suitable for administration by inhalation or insufflation.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates or sorbic acid).

For buccal administration, the composition may take the form of tablets or lozenges formulated in conventional manner.

The compounds of the invention may be formulated for parenteral administration by injection, including using conventional catheterization techniques or infusion. Formulations for injection may be presented in unit dosage form, e.g. in ampules or in multi-dose containers, with an added preservative. They may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g. sterile pyrogen-free water, before use.

When a product solution is required, it can be made by dissolving the isolated inclusion complex in water (or other aqueous medium) in an amount sufficient to generate a solution of the required strength for oral or parenteral administration to patients. The compounds may be formulated for fast dispersing dosage forms (fddf), which are designed to release the active ingredient in the oral cavity. These have often been formulated using rapidly soluble gelatin-based matrices. These dosage forms are well known and can be used to deliver a wide range of drugs. Most fast dispersing dosage forms utilize gelatin as a carrier or structure-forming agent. Typically, gelatin is used to give sufficient strength to the dosage form to prevent breakage during removal from packaging, but once placed in the mouth, the gelatin allows immediate dissolution of the dosage form. Alternatively, various starches are used to the same effect.

The compounds of the invention may also be formulated in rectal compositions such as suppositories or retention enemas, e.g. containing conventional suppository bases such as cocoa butter or other glycerides.

For intranasal administration or administration by inhalation, the compound of the invention is conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made e.g. from gelatin) for use in an inhaler or insufflator may be formulated containing a powder mix of a compound of the invention and a suitable powder base such as lactose or starch.

Aerosol formulations for treatment of the conditions referred to above (e.g. migraine) in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4 or 8 times, giving for example, 1, 2 or 3 doses each time.

A proposed daily dose of the compound of the invention for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

Assay methods are available to screen a substance for inhibition of cyclic nucleotide hydrolysis by the PDE 10 and the PDEs from other gene families. The cyclic nucleotide substrate concentration used in the assay is ⅓ of the Kₘ concentration, allowing for comparisons of IC₅₀ values across the different enzymes. PDE activity is measured using a Scintillation Proximity Assay (SPA)-based method as previously described (Fawcett et al., 2000). The effect of PDE inhibitors is determined by assaying a fixed amount of enzyme (PDEs 1-11) in the presence of varying substance concentrations and low substrate, such that the IC₅₀ approximates the Kᵢ (cGMP or cAMP in a 3:1 ratio unlabelled to [³H]-labeled at a concentration of ⅓ Km).). The final assay volume is made up to 100 μl with assay buffer [20 mM Tris-HCl pH 7.4, 5 mM MgCl₂, 1 mg/ml bovine serum albumin]. Reactions are initiated with enzyme, incubated for 30-60 min at 30° C. to give <30% substrate turnover and terminated with 50 μl yttrium silicate SPA beads (Amersham) (containing 3 mM of the respective unlabelled cyclic nucleotide for PDEs 9 and 11). Plates are re-sealed and shaken for 20 min, after which the beads were allowed to settle for 30 minutes in the dark and then counted on a TopCount plate reader (Packard, Meriden, Conn.). Radioactivity units can be converted to percent activity of an uninhibited control (100%), plotted against inhibitor concentration and inhibitor IC₅₀ values can be obtained using the “Fit Curve’ Microsoft Excel extension.

The following Examples illustrate the present invention. It is to be understood, however, that the invention, as fully described herein and as recited in the claims, is not intended to be limited by the details of the following Examples.

### EXAMPLES

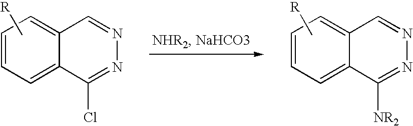

General Procedure for the Preparation of 4-Aminopthalazine Derivatives:

To a 0.2 M solution of 4-chloro-6,7-dimethoxyphthalazine (prepared as described in U.S. Pat. No. 4,370,328) in tetrahydrofuran is added an equal volume of saturated aqueous sodium bicarbonate. To the stirred mixture is added the NHR₂ amine component (amines prepared as in United States Publication Nos. 2006-0183763A1 (U.S. Ser. No. 11/257,179) and 2006-0019975A1 (U.S. Ser. No. 11/178,104) herein incorporated by reference in their entirety) and the resultant mixture is heated to a gentle reflux for 1-24 hours. The mixture is cooled to room temperature and partitioned between water and ethyl acetate. The organic portion is washed with brine, dried with magnesium sulfate, filtered and concentrated to yield the crude free-base product. The material is purified either by silica gel chromatography, or via formation of a hydrochloride salt and recrystallization.

The following prophetic Examples may be made by the General Procedure described above:
5-(4-methoxyphenyl)-1-(6,7-dimethoxyphthalazin-1-yl)piperidin-3-ol;
4,5,6,7-tetrahydro-5-(6,7-dimethoxyphthalazin-1-yl)-N-phenylthiazolo[5,4-c]pyridin-2-amine;
1-(4-((pyridin-4-yl)methoxy)piperidin-1-yl)-6,7-dimethoxyphthalazine;
1-(6,7-dimethoxyphthalazin-1-yl)-4-phenylpiperidin-4-ol;
4-benzyl-1-(6,7-dimethoxyphthalazin-1-yl)piperidin-4-ol;
[1-(6,7-dimethoxyphthalazin-1-yl)piperidin-4-yl](phenyl)methanone;
1-[4-(1H-123-benzotriazol-1-yl)piperidin-1-yl]-6,7-dimethoxyphthalazine;
6,7-dimethoxy-1-[4-(3-methylphenoxy)piperidin-1-yl]phthalazine;
6,7-dimethoxy-1-[4-(2-methylphenoxy)piperidin-1-yl]phthalazine;
6,7-dimethoxy-1-(4-pyridin-2-ylpiperidin-1-yl)phthalazine;
1-(4-benzylpiperidin-1-yl)-6,7-dimethoxyphthalazine;
1-[4-(benzyloxy)piperidin-1-yl]-6,7-dimethoxyphthalazine;
1-(6,7-dimethoxyphthalazin-1-yl)-4-phenylpiperidine-4-carbonitrile
1-(6,7-dimethoxyphthalazin-1-yl)-4-(3-fluorophenyl)piperidin-4-ol
6,7-dimethoxy-1-(4-phenoxypiperidin-1-yl)phthalazine;
6,7-dimethoxy-1-[4-(2-methoxyphenoxy)piperidin-1-yl]phthalazine;
6,7-dimethoxy-1-(3-phenyl piperidin-1-yl)phthalazine;
6,7-dimethoxy-1-(3-phenoxypiperidin-1-yl)phthalazine;
6,7-dimethoxy-1-[3-(2-methoxyphenyl)piperidin-1-yl]phthalazine;
1-[4-(3-ethyl-1,2,4-oxadiazol-5-yl)piperidin-1-yl]-6,7-dimethoxyphthalazine;
1-[3-(3-ethyl-1,2,4-oxadiazol-5-yl)piperidin-1-yl]-6,7-dimethoxyphthalazine;
2-{[1-(6,7-dimethoxyphthalazin-1-yl)piperidin-4-yl]oxy}benzonitrile
1-[4-(5-ethyl-1,2,4-oxadiazol-3-yl)piperidin-1-yl]-6,7-dimethoxyphthalazine;
6,7-dimethoxy-1-[4-(3-methoxyphenoxy)piperidin-1-yl]phthalazine;
6,7-dimethoxy-1-[4-(4-methoxyphenoxy)piperidin-1-yl]phthalazine;
1-[4-(2-fluorophenyl)piperidin-1-yl]-6,7-dimethoxyphthalazine;
6,7-dimethoxy-1-[3-(3-methyl-1,2,4-oxadiazol-5-yl)piperidin-1-yl]phthalazine;
6,7-dimethoxy-1-[3-(4-methylphenoxy)piperidin-1-yl]phthalazine;
1-[4-(3,5-dimethyl-4H-1,2,4-triazol-4-yl)piperidin-1-yl]-6,7-dimethoxyphthalazine;
6,7-dimethoxy-1-[3-(3-methylphenoxy)piperidin-1-yl]phthalazine;
6,7-dimethoxy-1-[3-(2-methoxyphenoxy)piperidin-1-yl]phthalazine;
6,7-dimethoxy-1-[3-(4-methoxyphenoxy)piperidin-1-yl]phthalazine;
6,7-dimethoxy-1-[4-(3-methyl-1,2,4-oxadiazol-5-yl)piperidin-1-yl]phthalazine;
6,7-dimethoxy-1-[3-(4-methyl-4H-1,2,4-triazol-3-yl)piperidin-1-yl]phthalazine;
1-(6,7-dimethoxyphthalazin-1-yl)-4-(4-fluorophenyl)piperidin-4-ol;
6,7-dimethoxy-1-[3-(3-methoxyphenyl)piperidin-1-yl]phthalazine;
1-(6,7-dimethoxyphthalazin-1-yl)-3-(24-dimethylphenyl)piperidin-3-ol;
1-(6,7-dimethoxyphthalazin-1-yl)-3-(2-ethylphenyl)piperidin-3-ol;
1-[1-(6,7-dimethoxyphthalazin-1-yl)-4-phenylpiperidin-4-yl]ethanone;
1-[4-(13-benzoxazol-2-yl)piperidin-1-yl]-6,7-dimethoxyphthalazine;
1-[3-(benzyloxy)piperidin-1-yl]-6,7-dimethoxyphthalazine;
1-[3-(13-benzoxazol-2-yl)piperidin-1-yl]-6,7-dimethoxyphthalazine;
[1-(6,7-dimethoxyphthalazin-1-yl)piperidin-3-](pyridin-3-yl)methanone;
[1-(6,7-dimethoxyphthalazin-1-yl)piperidin-3-yl](1-methyl-1H-imidazol-2-yl)methanone;
[1-(6,7-dimethoxyphthalazin-1-yl)piperidin-4-yl](phenyl)methanol;
[1-(6,7-dimethoxyphthalazin-1-yl)piperidin-4-yl](pyridin-2-yl)methanol;
[1-(6,7-dimethoxyphthalazin-1-yl)piperidin-4-yl](pyridin-3-yl)methanol;
[1-(6,7-dimethoxyphthalazin-1-yl)piperidin-4-yl](1-methyl-1H-imidazol-2-yl)methanol;
6,7-dimethoxy-1-[3-(5-methyl-1,2,4-oxadiazol-3-yl)piperidin-1-yl]phthalazine;
1′-(6,7-dimethoxyphthalazin-1-yl)-34-dihydro-2H-spiro[isoquinoline-14′-piperidine]
3-[1-(6,7-dimethoxyphthalazin-1-yl)piperidin-4-yl]phenol;
1-[3-(3-tert-butyl-1,2,4-oxadiazol-5-yl)piperidin-1-yl]-6,7-dimethoxyphthalazine;
1-[3-(4-chlorophenyl)piperidin-1-yl]-6,7-dimethoxyphthalazine;
6,7-dimethoxy-1-(4-pyridin-4-ylpiperidin-1-yl)phthalazine;
1-{3-[(2-fluorophenoxy)methyl]piperidin-1-yl}-6,7-dimethoxyphthalazine;
[1-(6,7-dimethoxyphthalazin-1-yl)piperidin-3-yl](phenyl)methanone;
[1-(6,7-dimethoxyphthalazin-1-yl)piperidin-3-yl](pyridin-2-yl)methanone;
6,7-dimethoxy-1-[3-(5-methyl-1,3,4-oxadiazol-2-yl)piperidin-1-yl]phthalazine;
6,7-dimethoxy-1-[4-(1H-pyrazol-5-yl)piperidin-1-yl]phthalazine;
[4-benzyl-1-(6,7-dimethoxyphthalazin-1-yl)piperidin-4-yl]methanol;
6,7-dimethoxy-1-[4-(2-methylphenyl)piperidin-1-yl]phthalazine;
1-(6,7-dimethoxyphthalazin-1-yl)-4-(4-methylphenyl)piperidin-4-ol;
6,7-dimethoxy-1-[3-(phenoxymethyl)piperidin-1-yl]phthalazine;
6,7-dimethoxy-1-(3-{[(2-methylpyridin-3-yl)oxy]methyl}piperidin-1-yl)phthalazine;
6,7-dimethoxy-1-(3-{[(6-methylpyridin-3-yl)oxy]methyl}piperidin-1-yl)phthalazine;
3-{[1-(6,7-dimethoxyphthalazin-1-yl)piperidin-3-yl]methoxy}pyridin-2-amine
1-(6,7-dimethoxyphthalazin-1-yl)-3-[(2-methyl-1H-imidazol-1-yl)methyl]piperidin-3-ol;
6,7-dimethoxy-1-{4-[5-(methoxymethyl)-1,3,4-oxadiazol-2-yl]piperidin-1-yl}phthalazine;
1-[4-(5-isobutyl-1,3,4-oxadiazol-2-yl)piperidin-1-yl]-6,7-dimethoxyphthalazine;
1-[3-(5-cyclopropyl-1,3,4-oxadiazol-2-yl)piperidin-1-yl]-6,7-dimethoxyphthalazine;
6,7-dimethoxy-1-{3-[5-(methoxymethyl)-1,3,4-oxadiazol-2-yl]piperidin-1-yl}phthalazine;
1-{5-[1-(6,7-dimethoxyphthalazin-1-yl)piperidin-3-yl]-1,3,4-oxadiazol-2-yl}-N,N-dimethylmethanamine
6,7-dimethoxy-1-{4-[(6-methylpyridazin-3-yl)methyl]piperidin-1-yl}phthalazine;
6,7-dimethoxy-1-[4-(pyrimidin-2-ylmethyl)piperidin-1-yl]phthalazine;
1-(6,7-dimethoxyphthalazin-1-yl)-3-(1H-pyrazol-1-ylmethyl)piperidin-3-ol;
6,7-dimethoxy-1-[4-(2-methylpyrimidin-4-yl)piperidin-1-yl]phthalazine;
6,7-dimethoxy-1-[3-(pyridin-2-ylmethyl)piperidin-1-yl]phthalazine;
6,7-dimethoxy-1-[3-(pyrimidin-2-ylmethyl)piperidin-1-yl]phthalazine;
(3-{[1-(6,7-dimethoxyphthalazin-1-yl)piperidin-3-yl]methyl}phenyl)methanol;
6,7-dimethoxy-1-{3-[2-(methoxymethyl)pyrimidin-4-yl]piperidin-1-yl}phthalazine;
6,7-dimethoxy-1-(3-pyrimidin-4-ylpiperidin-1-yl)phthalazine;
4-[1-(6,7-dimethoxyphthalazin-1-yl)piperidin-4-yl]-N-ethylpyrimidin-2-amine
6,7-dimethoxy-1-(4-pyrimidin-4-ylpiperidin-1-yl)phthalazine;
1-(6,7-dimethoxyphthalazin-1-yl)-4-[(2-methyl-1H-imidazol-1-yl)methyl]piperidin-4-ol;
6,7-dimethoxy-1-(4-[1,2,4]triazolo[15-a]pyrimidin-7-ylpiperidin-1-yl)phthalazine;
1-[4-(2-cyclopropylpyrimidin-4-yl)piperidin-1-yl]-6,7-dimethoxyphthalazine;
1-[4-(5-cyclopropyl-1,3,4-oxadiazol-2-yl)piperidin-1-yl]-6,7-dimethoxyphthalazine;
6,7-dimethoxy-1-[4-(pyrazin-2-ylmethyl)piperidin-1-yl]phthalazine;
3-[1-(6,7-dimethoxyphthalazin-1-yl)piperidin-4-yl]benzamide
4-[1-(6,7-dimethoxyphthalazin-1-yl)piperidin-4-yl]benzamide
6,7-dimethoxy-1-(4-pyrimidin-2-ylpiperidin-1-yl)phthalazine;
1-[3-(3-chlorobenzyl)piperidin-1-yl]-6,7-dimethoxyphthalazine;
6,7-dimethoxy-1-[3-(pyrimidin-5-ylmethyl)piperidin-1-yl]phthalazine;
4-[1-(6,7-dimethoxyphthalazin-1-yl)piperidin-4-yl]-N,N-dimethylpyrimidin-2-amine
6,7-dimethoxy-1-[4-(pyrimidin-5-ylmethyl)piperidin-1-yl]phthalazine;
1-[3-(1H-benzimidazol-2-yl)piperidin-1-yl]-6,7-dimethoxyphthalazine;
1-[4-(4-fluorobenzyl)piperidin-1-yl]-6,7-dimethoxyphthalazine;
6,7-dimethoxy-1-[3-(3-methylphenyl)piperidin-1-yl]phthalazine;
1-[4-(2-fluorobenzyl)piperidin-1-yl]-6,7-dimethoxyphthalazine;
6,7-dimethoxy-1-{3-[(2-methyl-1H-imidazol-1-yl)methyl]piperidin-1-yl}phthalazine;
6,7-dimethoxy-1-[4-(1H-pyrazol-1-ylmethyl)piperidin-1-yl]phthalazine;
1-[4-(3-cyclopropyl-1,2,4-oxadiazol-5-yl)piperidin-1-yl]-6,7-dimethoxyphthalazine;
1-[3-(5-cyclopropyl-4H-1,2,4-triazol-3-yl)piperidin-1-yl]-6,7-dimethoxyphthalazine;
1-(6,7-dimethoxyphthalazin-1-yl)-4-(2-fluoro-5-methylphenyl)piperidin-4-ol;
1-[4-(2-fluorophenoxy)piperidin-1-yl]-6,7-dimethoxyphthalazine;
1-{3-[(4-fluorophenoxy)methyl]piperidin-1-yl}-6,7-dimethoxyphthalazine;
6,7-dimethoxy-1-[4-(pyridin-2-ylmethoxy)piperidin-1-yl]phthalazine;
6,7-dimethoxy-1-{4-[5-(methoxymethyl)-1,2,4-oxadiazol-3-yl]piperidin-1-yl}phthalazine;
6,7-dimethoxy-1-[4-(3-methoxyphenyl)piperidin-1-yl]phthalazine;
1-[4-(3,5-difluorophenyl)piperidin-1-yl]-6,7-dimethoxyphthalazine;
1-[4-(3-fluorophenoxy)piperidin-1-yl]-6,7-dimethoxyphthalazine;
1-[4-(4-fluorophenoxy)piperidin-1-yl]-6,7-dimethoxyphthalazine;
4-[1-(6,7-dimethoxyphthalazin-1-yl)piperidin-4-yl]benzonitrile
1′-(6,7-dimethoxyphthalazin-1-yl)spiro[chromene-2,4′-piperidine]
1-[3-(1H-imidazol-2-yl)piperidin-1-yl]-6,7-dimethoxyphthalazine;
1′-(6,7-dimethoxyphthalazin-1-yl)-34-dihydrospiro[isochromene-1,4′-piperidine]
N-[1-(6,7-dimethoxyphthalazin-1-yl)piperidin-4-yl]pyrimidin-2-amine
N-[1-(6,7-dimethoxyphthalazin-1-yl)piperidin-4-yl]pyridin-2-amine
N-benzyl-1-(6,7-dimethoxyphthalazin-1-yl)-N-methylpiperidin-4-amine
6,7-dimethoxy-1-[4-(4-methylbenzyl)piperidin-1-yl]phthalazine;
1-[4-(4-chlorophenyl)-4-methylpiperidin-1-yl]-6,7-dimethoxyphthalazine;
1′-(6,7-dimethoxyphthalazin-1-yl)spiro[indole-34′-piperidin]-2(1H)-one;
4-phenyl-1-(5,6,7-trimethoxyphthalazin-1-yl)piperidin-4-ol;
4-benzyl-1-(5,6,7-trimethoxyphthalazin-1-yl)piperidin-4-ol;
phenyl[1-(5,6,7-trimethoxyphthalazin-1-yl)piperidin-4-yl]methanone;
1-[4-(1H-123-benzotriazol-1-yl)piperidin-1-yl]-5,6,7-trimethoxyphthalazine;
5,6,7-trimethoxy-1-[4-(3-methylphenoxy)piperidin-1-yl]phthalazine;
5,6,7-trimethoxy-1-[4-(2-methylphenoxy)piperidin-1-yl]phthalazine;
5,6,7-trimethoxy-1-(4-pyridin-2-ylpiperidin-1-yl)phthalazine;
1-(4-benzylpiperidin-1-yl)-5,6,7-trimethoxyphthalazine;
1-[4-(benzyloxy)piperidin-1-yl]-5,6,7-trimethoxyphthalazine;
4-phenyl-1-(5,6,7-trimethoxyphthalazin-1-yl)piperidine-4-carbonitrile
4-(3-fluorophenyl)-1-(5,6,7-trimethoxyphthalazin-1-yl)piperidin-4-ol;
5,6,7-trimethoxy-1-(4-phenoxypiperidin-1-yl)phthalazine;
5,6,7-trimethoxy-1-[4-(2-methoxyphenoxy)piperidin-1-yl]phthalazine;
5,6,7-trimethoxy-1-(3-phenylpiperidin-1-yl)phthalazine;
5,6,7-trimethoxy-1-(3-phenoxypiperidin-1-yl)phthalazine;
5,6,7-trimethoxy-1-[3-(2-methoxyphenyl)piperidin-1-yl]phthalazine;
1-[4-(3-ethyl-1,2,4-oxadiazol-5-yl)piperidin-1-yl]-5,6,7-trimethoxyphthalazine;
1-[3-(3-ethyl-1,2,4-oxadiazol-5-yl)piperidin-1-yl]-5,6,7-trimethoxyphthalazine;
2-{[1-(5,6,7-trimethoxyphthalazin-1-yl)piperidin-4-yl]oxy}benzonitrile
1-[4-(5-ethyl-1,2,4-oxadiazol-3-yl)piperidin-1-yl]-5,6,7-trimethoxyphthalazine;
5,6,7-trimethoxy-1-[4-(3-methoxyphenoxy)piperidin-1-yl]phthalazine;
5,6,7-trimethoxy-1-[4-(4-methoxyphenoxy)piperidin-1-yl]phthalazine;
1-[4-(2-fluorophenyl)piperidin-1-yl]-5,6,7-trimethoxyphthalazine;
5,6,7-trimethoxy-1-[3-(3-methyl-1,2,4-oxadiazol-5-yl)piperidin-1-yl]phthalazine;
5,6,7-trimethoxy-1-[3-(4-methylphenoxy)piperidin-1-yl]phthalazine;
1-[4-(3,5-dimethyl-4H-1,2,4-triazol-4-yl)piperidin-1-yl]-5,6,7-trimethoxyphthalazine;
5,6,7-trimethoxy-1-[3-(3-methylphenoxy)piperidin-1-yl]phthalazine;
5,6,7-trimethoxy-1-[3-(2-methoxyphenoxy)piperidin-1-yl]phthalazine;
5,6,7-trimethoxy-1-[3-(4-methoxyphenoxy)piperidin-1-yl]phthalazine;
5,6,7-trimethoxy-1-[4-(3-methyl-1,2,4-oxadiazol-5-yl)piperidin-1-yl]phthalazine;
5,6,7-trimethoxy-1-[3-(4-methyl-4H-1,2,4-triazol-3-yl)piperidin-1-yl]phthalazine;
4-(4-fluorophenyl)-1-(5,6,7-trimethoxyphthalazin-1-yl)piperidin-4-ol;
5,6,7-trimethoxy-1-[3-(3-methoxyphenyl)piperidin-1-yl]phthalazine;
3-(24-dimethylphenyl)-1-(5,6,7-trimethoxyphthalazin-1-yl)piperidin-3-ol;
3-(2-ethylphenyl)-1-(5,6,7-trimethoxyphthalazin-1-yl)piperidin-3-ol;
1-[4-phenyl-1-(5,6,7-trimethoxyphthalazin-1-yl)piperidin-4-yl]ethanone;
1-[4-(13-benzoxazol-2-yl)piperidin-1-yl]-5,6,7-trimethoxyphthalazine;
1-[3-(benzyloxy)piperidin-1-yl]-5,6,7-trimethoxyphthalazine;
1-[3-(13-benzoxazol-2-yl)piperidin-1-yl]-5,6,7-trimethoxyphthalazine;
pyridin-3-yl[1-(5,6,7-trimethoxyphthalazin-1-yl)piperidin-3-yl]methanone;
(1-methyl-1H-imidazol-2-yl)[1-(5,6,7-trimethoxyphthalazin-1-yl)piperidin-3-yl]methanone;
phenyl[1-(5,6,7-trimethoxyphthalazin-1-yl)piperidin-4-yl]methanol;
pyridin-2-yl[1-(5,6,7-trimethoxyphthalazin-1-yl)piperidin-4-yl]methanol;
pyridin-3-yl[1-(5,6,7-trimethoxyphthalazin-1-yl)piperidin-4-yl]methanol;
(1-methyl-1H-imidazol-2-yl)[1-(5,6,7-trimethoxyphthalazin-1-yl)piperidin-4-yl]methanol;
5,6,7-trimethoxy-1-[3-(5-methyl-1,2,4-oxadiazol-3-yl)piperidin-1-yl]phthalazine;
1′-(5,6,7-trimethoxyphthalazin-1-yl)-34-dihydro-2H-spiro[isoquinoline-14′-piperidine];
3-[1-(5,6,7-trimethoxyphthalazin-1-yl)piperidin-4-yl]phenol;
1-[3-(3-tert-butyl-1,2,4-oxadiazol-5-yl)piperidin-1-yl]-5,6,7-trimethoxyphthalazine;
1-[3-(4-chlorophenyl)piperidin-1-yl]-5,6,7-trimethoxyphthalazine;
5,6,7-trimethoxy-1-(4-pyridin-4-ylpiperidin-1-yl)phthalazine;
1-{3-[(2-fluorophenoxy)methyl]piperidin-1-yl}-5,6,7-trimethoxyphthalazine;
phenyl[1-(5,6,7-trimethoxyphthalazin-1-yl)piperidin-3-yl]methanone;
pyridin-2-yl[1-(5,6,7-trimethoxyphthalazin-1-yl)piperidin-3-yl]methanone;
5,6,7-trimethoxy-1-[3-(5-methyl-1,3,4-oxadiazol-2-yl)piperidin-1-yl]phthalazine;
5,6,7-trimethoxy-1-[4-(1H-pyrazol-5-yl)piperidin-1-yl]phthalazine;
[4-benzyl-1-(5,6,7-trimethoxyphthalazin-1-yl)piperidin-4-yl]methanol;
5,6,7-trimethoxy-1-[4-(2-methylphenyl)piperidin-1-yl]phthalazine;
4-(4-methylphenyl)-1-(5,6,7-trimethoxyphthalazin-1-yl)piperidin-4-ol;
5,6,7-trimethoxy-1-[3-(phenoxymethyl)piperidin-1-yl]phthalazine;
5,6,7-trimethoxy-1-(3-{[(2-methylpyridin-3-yl)oxy]methyl}piperidin-1-yl)phthalazine;
5,6,7-trimethoxy-1-(3-{[(6-methylpyridin-3-yl)oxy]methyl}piperidin-1-yl)phthalazine;
3-{[1-(5,6,7-trimethoxyphthalazin-1-yl)piperidin-3-yl]methoxy}pyridin-2-amine;
3-[(2-methyl-1H-imidazol-1-yl)methyl]-1-(5,6,7-trimethoxyphthalazin-1-yl)piperidin-3-ol;
5,6,7-trimethoxy-1-{4-[5-(methoxymethyl)-1,3,4-oxadiazol-2-yl]piperidin-1-yl}phthalazine;
1-[4-(5-isobutyl-1,3,4-oxadiazol-2-yl)piperidin-1-yl]-5,6,7-trimethoxyphthalazine;
1-[3-(5-cyclopropyl-1,3,4-oxadiazol-2-yl)piperidin-1-yl]-5,6,7-trimethoxyphthalazine;
5,6,7-trimethoxy-1-{3-[5-(methoxymethyl)-1,3,4-oxadiazol-2-yl]piperidin-1-yl}phthalazine;
N,N-dimethyl-1-{5-[1-(5,6,7-trimethoxyphthalazin-1-yl)piperidin-3-yl]-1,3,4-oxadiazol-2-yl}methanamine
5,6,7-trimethoxy-1-{4-[(6-methylpyridazin-3-yl)methyl]piperidin-1-yl}phthalazine;
5,6,7-trimethoxy-1-[4-(pyrimidin-2-ylmethyl)piperidin-1-yl]phthalazine;
3-(1H-pyrazol-1-ylmethyl)-1-(5,6,7-trimethoxyphthalazin-1-yl)piperidin-3-ol;
5,6,7-trimethoxy-1-[4-(2-methylpyrimidin-4-yl)piperidin-1-yl]phthalazine;
5,6,7-trimethoxy-1-[3-(pyridin-2-ylmethyl)piperidin-1-yl]phthalazine;
5,6,7-trimethoxy-1-[3-(pyrimidin-2-ylmethyl)piperidin-1-yl]phthalazine;
(3-{[1-(5,6,7-trimethoxyphthalazin-1-yl)piperidin-3-yl]methyl}phenyl)methanol;
5,6,7-trimethoxy-1-{3-[2-(methoxymethyl)pyrimidin-4-yl]piperidin-1-yl}phthalazine;
5,6,7-trimethoxy-1-(3-pyrimidin-4-ylpiperidin-1-yl)phthalazine;
N-ethyl-4-[1-(5,6,7-trimethoxyphthalazin-1-yl)piperidin-4-yl]pyrimidin-2-amine
5,6,7-trimethoxy-1-(4-pyrimidin-4-ylpiperidin-1-yl)phthalazine;
4-[(2-methyl-1H-imidazol-1-yl)methyl]-1-(5,6,7-trimethoxyphthalazin-1-yl)piperidin-4-ol;
5,6,7-trimethoxy-1-(4-[1,2,4]triazolo[15-a]pyrimidin-7-ylpiperidin-1-yl)phthalazine;
1-[4-(2-cyclopropylpyrimidin-4-yl)piperidin-1-yl]-5,6,7-trimethoxyphthalazine;
1-[4-(5-cyclopropyl-1,3,4-oxadiazol-2-yl)piperidin-1-yl]-5,6,7-trimethoxyphthalazine;
5,6,7-trimethoxy-1-[4-(pyrazin-2-ylmethyl)piperidin-1-yl]phthalazine;
3-[1-(5,6,7-trimethoxyphthalazin-1-yl)piperidin-4-yl]benzamide;
4-[1-(5,6,7-trimethoxyphthalazin-1-yl)piperidin-4-yl]benzamide;
5,6,7-trimethoxy-1-(4-pyrimidin-2-ylpiperidin-1-yl)phthalazine;
1-[3-(3-chlorobenzyl)piperidin-1-yl]-5,6,7-trimethoxyphthalazine;
5,6,7-trimethoxy-1-[3-(pyrimidin-5-ylmethyl)piperidin-1-yl]phthalazine;
N,N-dimethyl-4-[1-(5,6,7-trimethoxyphthalazin-1-yl)piperidin-4-yl]pyrimidin-2-amine
5,6,7-trimethoxy-1-[4-(pyrimidin-5-ylmethyl)piperidin-1-yl]phthalazine;
1-[3-(1H-benzimidazol-2-yl)piperidin-1-yl]-5,6,7-trimethoxyphthalazine;
1-[4-(4-fluorobenzyl)piperidin-1-yl]-5,6,7-trimethoxyphthalazine;
5,6,7-trimethoxy-1-[3-(3-methylphenyl)piperidin-1-yl]phthalazine;
1-[4-(2-fluorobenzyl)piperidin-1-yl]-5,6,7-trimethoxyphthalazine;
5,6,7-trimethoxy-1-{3-[(2-methyl-1H-imidazol-1-yl)methyl]piperidin-1-yl}phthalazine;
5,6,7-trimethoxy-1-[4-(1H-pyrazol-1-ylmethyl)piperidin-1-yl]phthalazine;
1-[4-(3-cyclopropyl-1,2,4-oxadiazol-5-yl)piperidin-1-yl]-5,6,7-trimethoxyphthalazine;
1-[3-(5-cyclopropyl-4H-1,2,4-triazol-3-yl)piperidin-1-yl]-5,6,7-trimethoxyphthalazine;
4-(2-fluoro-5-methylphenyl)-1-(5,6,7-trimethoxyphthalazin-1-yl)piperidin-4-ol;
1-[4-(2-fluorophenoxy)piperidin-1-yl]-5,6,7-trimethoxyphthalazine;
1-{3-[(4-fluorophenoxy)methyl]piperidin-1-yl}-5,6,7-trimethoxyphthalazine;
5,6,7-trimethoxy-1-[4-(pyridin-2-ylmethoxy)piperidin-1-yl]phthalazine;
5,6,7-trimethoxy-1-{4-[5-(methoxymethyl)-1,2,4-oxadiazol-3-yl]piperidin-1-yl}phthalazine;
5,6,7-trimethoxy-1-[4-(3-methoxyphenyl)piperidin-1-yl]phthalazine;
1-[4-(3,5-difluorophenyl)piperidin-1-yl]-5,6,7-trimethoxyphthalazine;
1-[4-(3-fluorophenoxy)piperidin-1-yl]-5,6,7-trimethoxyphthalazine;
1-[4-(4-fluorophenoxy)piperidin-1-yl]-5,6,7-trimethoxyphthalazine;
4-[1-(5,6,7-trimethoxyphthalazin-1-yl)piperidin-4-yl]benzonitrile
1′-(5,6,7-trimethoxyphthalazin-1-yl)spiro[chromene-24′-piperidine]
1-[3-(1H-imidazol-2-yl)piperidin-1-yl]-5,6,7-trimethoxyphthalazine;
1′-(5,6,7-trimethoxyphthalazin-1-yl)-34-dihydrospiro[isochromene-1,4′-piperidine]
N-[1-(5,6,7-trimethoxyphthalazin-1-yl)piperidin-4-yl]pyrimidin-2-amine
N-[1-(5,6,7-trimethoxyphthalazin-1-yl)piperidin-4-yl]pyridin-2-amine
N-benzyl-N-methyl-1-(5,6,7-trimethoxyphthalazin-1-yl)piperidin-4-amine
5,6,7-trimethoxy-1-[4-(4-methylbenzyl)piperidin-1-yl]phthalazine;
1-[4-(4-chlorophenyl)-4-methylpiperidin-1-yl]-5,6,7-trimethoxyphthalazine; and
1′-(5,6,7-trimethoxyphthalazin-1-yl)spiro[indole-34′-piperidin]-2(1H)-one; and pharmaceutical salts thereof.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed:

1. A compound of the formula I: 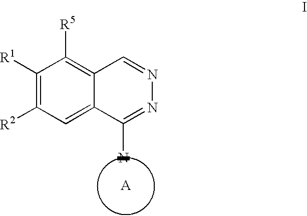
or a pharmaceutically acceptable salt thereof,
wherein ring A is a 5 or 6 membered heterocyclic ring substituted by at least one R⁶ and at least one R⁷;
wherein R¹, R² and R⁵ are each independently H, halogen, —CN, —COON, —COOR³, —CONR³R⁴, —COR²⁰, —NR³R⁴, —NHCOR²⁰, —OH, (C₆-C₁₀)aryl, (5-10) membered heteroaryl, (C₁-C₆)alkyl, (C₁-C₆)haloalkyl (C₂-C₆)alkenyl, (C₂-C₆)alkynyl, (C₁-C₆)alkoxy, (C₂-C₆)alkenyloxy or (C₃-C₈)cycloalkyl; or, when R¹, R² and R⁵ are independently (C₁-C₆)alkoxy, (C₂-C₆)alkenyloxy, (C₁-C₆)alkyl, (C₂-C₆)alkenyl or (C₂-C₆)alkynyl, R¹ and R² or R¹ and R⁵ may optionally be connected to form a 5 to 8 membered ring;
wherein R³ and R⁴ are each independently H, (C₁-C₆)alkyl (5-10 membered)heteroaryl or (C₆-C₁₀)aryl, wherein said heteroaryl or aryl may be optionally substituted with one or more (C₁-C₆)alkyl or halo groups;
wherein each R⁶ is independently H, halogen, —COOR³, —CONR³R⁴, —COR²⁰, —NR³R⁴, —OH, (C₁-C₆)hydroxyalkyl —HNCOOR³, —CN, —HNCONHR⁴, (C₁-C₆)alkyl, (C₁-C₆)alkoxy, (C₆-C₁₀)aryl, —O—(C₁-C₆)alkylene-(5-8 membered)heteroaryl, —O—(C₁-C₆)alkylene-(C₆-C₁₀)aryl, —(C₁-C₆)alkylene-O-(5-8 membered)heteroaryl, —(C₁-C₆)alkylene-O—(C₆-C₁₀)aryl or 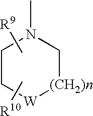
wherein n is 0 or 1;
W is carbon, oxygen or NR⁸, wherein R⁸ is hydrogen or (C₁-C₆)alkyl, and when W is carbon, it may be optionally substituted by halogen, —CN, —COOH, —COOR³, —CONR³R⁴, —COR²⁰, —NR³R⁴, —NHCOR²⁰, —OH, (C₆-C₁₀)aryl, (5-10) membered heteroaryl, (C₁-C₆)alkyl, (C₁-C₆)haloalkyl (C₂-C₆)alkenyl, (C₂-C₆)alkynyl, (C₁-C₆)alkyloxy, (C₂-C₆)alkenyloxy or (C₃-C₈)cycloalkyl; and wherein said alkyl, aryl or heteroaryl of R⁶ may be optionally substituted by (C₁-C₈)alkyl, (C₃-C₈)cycloalkyl, (C₁-C₈)alkoxy, halogen, —OH, and halo(C₁-C₈)alkyl;
wherein R⁹ and R¹⁰ are independently hydrogen or (C₁-C₆)alkyl;
or R⁹ and R¹⁰ may optionally combine to form a cyclic ring;
wherein each R⁷ is independently R¹¹, —R¹⁸—R¹¹ or —OR¹¹;
wherein R¹¹ is hydrogen, phenyl, naphthyl, or a 5- to 6-membered heteroaryl ring, optionally fused to a benzo group or heteroaryl ring, containing from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈)alkyl, (C₃-C₈)cycloalkyl, (C₁-C₈)alkoxy, halogen, —CN, —OH, (C₆-C₁₀)aryl, (5-10) membered heteroaryl, halo(C₁-C₈)alkyl, (C₁-C₈)hydroxyalkyl, (C₁-C₈)alkoxy-(C₁-C₈)alkyl, (C₃-C₈)hydroxycycloalkyl, (C₃-C₈)cycloalkoxy, (C₁-C₈)alkoxy-(C₃-C₈)cycloalkyl, (C₃-C₈)heterocycloalkyl, (C₃-C₈)hydroxyheterocycloalkyl, and (C₁-C₈)alkoxy-heterocycloalkyl, wherein each cycloalkyl or heterocycloalkyl moiety may be independently substituted with from one to three halogens, (C₁-C₆)alkyl or benzyl groups; or
when R¹¹ is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR¹⁴R¹⁵ or —(C₀-C₆)alkylene-NR¹⁴R¹⁵, wherein R¹⁴ and R¹⁵ are each independently selected from hydrogen, (C₁-C₈)alkyl and benzyl, or R¹⁴ and R¹⁵ together with the nitrogen to which they are attached form a (5-7) membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; (c) —(CH₂)ᵥNCOR¹⁶R¹⁷, wherein v is zero, one, two or three and —COR¹⁶ and R¹⁷ taken together with the nitrogen to which they are attached may form a 4- to 6-membered lactam ring; or
when R¹¹ is heteroaryl, it may be optionally fused to ring A and optionally substituted with —NR¹²R¹³;
wherein R¹², R¹³, R¹⁶ and R¹⁷ are each independently hydrogen, (C₁-C₆)alkyl, and (C₆-C₁₀)aryl;
wherein R¹⁸ is (C₁-C₃) alkylene or —N(R₁₉)—; wherein said alkylene may be optionally substituted by (C₁-C₈)alkyl, (C₃-C₈)cycloalkyl, (C₁-C₈)alkoxy, halogen, —OH, or halo(C₁-C₈)alkyl;
R¹⁹ is hydrogen or (C₁-C₆)alkyl; and
wherein each R²⁰ is independently (C₁-C₈)alkyl, (C₃-C₈)cycloalkyl (C₁-C₈)alkoxy, halo(C₁-C₈)alkyl, (C₁-C₈)hydroxyalkyl, (C₁-C₈)alkoxy-(C₁-C₈)alkyl, (C₃-C₈)hydroxycycloalkyl, (C₃-C₈)cycloalkoxy, (C₁-C₈)alkoxy-(C₃-C₈)cycloalkyl, (C₃-C₈)heterocycloalkyl, (C₆-C₁₀)aryl or (5-10) membered heteroaryl.

2. The compound of claim 1, wherein ring A is piperidine or pyrrolidine.

3. The compound of claim 2, wherein R¹ and R² are each independently (C₁-C₄)alkoxy.

4. The compound of claim 2, wherein, R⁷ is R¹¹ and R¹¹ is phenyl optionally substituted by (C₁-C₆)alkoxy, (C₁-C₅)alkyl, —CN, —OH, phenyl or (C₁-C₆)alkoxy substituted with 1 to 3 halogens.

5. The compound of claim 2, wherein R₇ is —OR¹¹ and R¹¹ is naphthyl or naphthyl substituted by (C₁-C₆)alkoxy.

6. The compound of claim 2, wherein R⁷ is —OR¹¹ and R¹¹ is 5 or 6 membered heteroaryl.

7. The compound of claim 2, wherein R⁶ is (C₁-C₆)alkoxy or —OH.

8. The compound of claim 2, wherein, R⁶ is —NR³R⁴, and R³ and R⁴ are each independently (C₁-C₃)alkyl.

9. The compound of claim 2, wherein R¹ and R² are each independently (C₁-C₆)alkoxy, R⁷ is R¹¹ and R¹¹ is phenyl or substituted phenyl and R⁶ is (C₁-C₆)alkoxy or —OH.

10. The compound of claim 1, wherein R⁶ and R⁷ can not both be hydrogen.

11. The compound of claim 1, wherein said compound is selected from the group consisting of:
5-(4-methoxyphenyl)-1-(6,7-dimethoxyphthalazin-1-yl)piperidin-3-ol;
4,5,6,7-tetrahydro-5-(6,7-dimethoxyphthalazin-1-yl)-N-phenylthiazolo[5,4-c]pyridin-2-amine;
1-(4-((pyridin-4-yl)methoxy)piperidin-1-yl)-6,7-dimethoxyphthalazine;
1-(6,7-dimethoxyphthalazin-1-yl)-4-phenylpiperidin-4-ol;
4-benzyl-1-(6,7-dimethoxyphthalazin-1-yl)piperidin-4-ol;
[1-(6,7-dimethoxyphthalazin-1-yl)piperidin-4-yl](phenyl)methanone;
1-[4-(1H-123-benzotriazol-1-yl)piperidin-1-yl]-6,7-dimethoxyphthalazine;
6,7-dimethoxy-1-[4-(3-methylphenoxy)piperidin-1-yl]phthalazine;
6,7-dimethoxy-1-[4-(2-methylphenoxy)piperidin-1-yl]phthalazine;
6,7-dimethoxy-1-(4-pyridin-2-ylpiperidin-1-yl)phthalazine;
1-(4-benzylpiperidin-1-yl)-6,7-dimethoxyphthalazine;
1-[4-(benzyloxy)piperidin-1-yl]-6,7-dimethoxyphthalazine;
1-(6,7-dimethoxyphthalazin-1-yl)-4-phenylpiperidine-4-carbonitrile
1-(6,7-dimethoxyphthalazin-1-yl)-4-(3-fluorophenyl)piperidin-4-ol
6,7-dimethoxy-1-(4-phenoxypiperidin-1-yl)phthalazine;
6,7-dimethoxy-1-[4-(2-methoxyphenoxy)piperidin-1-yl]phthalazine;
6,7-dimethoxy-1-(3-phenylpiperidin-1-yl)phthalazine;
6,7-dimethoxy-1-(3-phenoxypiperidin-1-yl)phthalazine;
6,7-dimethoxy-1-[3-(2-methoxyphenyl)piperidin-1-yl]phthalazine;
1-[4-(3-ethyl-1,2,4-oxadiazol-5-yl)piperidin-1-yl]-6,7-dimethoxyphthalazine;
1-[3-(3-ethyl-1,2,4-oxadiazol-5-yl)piperidin-1-yl]-6,7-dimethoxyphthalazine;
2-{[1-(6,7-dimethoxyphthalazin-1-yl)piperidin-4-yl]oxy}benzonitrile
1-[4-(5-ethyl-1,2,4-oxadiazol-3-yl)piperidin-1-yl]-6,7-dimethoxyphthalazine;
6,7-dimethoxy-1-[4-(3-methoxyphenoxy)piperidin-1-yl]phthalazine;
6,7-dimethoxy-1-[4-(4-methoxyphenoxy)piperidin-1-yl]phthalazine;
1-[4-(2-fluorophenyl)piperidin-1-yl]-6,7-dimethoxyphthalazine;
6,7-dimethoxy-1-[3-(3-methyl-1,2,4-oxadiazol-5-yl)piperidin-1-yl]phthalazine;
6,7-dimethoxy-1-[3-(4-methylphenoxy)piperidin-1-yl]phthalazine;
1-[4-(3,5-dimethyl-4H-1,2,4-triazol-4-yl)piperidin-1-yl]-6,7-dimethoxyphthalazine;
6,7-dimethoxy-1-[3-(3-methylphenoxy)piperidin-1-yl]phthalazine;
6,7-dimethoxy-1-[3-(2-methoxyphenoxy)piperidin-1-yl]phthalazine;
6,7-dimethoxy-1-[3-(4-methoxyphenoxy)piperidin-1-yl]phthalazine;
6,7-dimethoxy-1-[4-(3-methyl-1,2,4-oxadiazol-5-yl)piperidin-1-yl]phthalazine;
6,7-dimethoxy-1-[3-(4-methyl-4H-1,2,4-triazol-3-yl)piperidin-1-yl]phthalazine;
1-(6,7-dimethoxyphthalazin-1-yl)-4-(4-fluorophenyl)piperidin-4-ol;
6,7-dimethoxy-1-[3-(3-methoxyphenyl)piperidin-1-yl]phthalazine;
1-(6,7-dimethoxyphthalazin-1-yl)-3-(24-dimethylphenyl)piperidin-3-ol;
1-(6,7-dimethoxyphthalazin-1-yl)-3-(2-ethylphenyl)piperidin-3-ol;
1-[1-(6,7-dimethoxyphthalazin-1-yl)-4-phenylpiperidin-4-yl]ethanone;
1-[4-(13-benzoxazol-2-yl)piperidin-1-yl]-6,7-dimethoxyphthalazine;
1-[3-(benzyloxy)piperidin-1-yl]-6,7-dimethoxyphthalazine;
1-[3-(13-benzoxazol-2-yl)piperidin-1-yl]-6,7-dimethoxyphthalazine;
[1-(6,7-dimethoxyphthalazin-1-yl)piperidin-3-yl](pyridin-3-yl)methanone;
[1-(6,7-dimethoxyphthalazin-1-yl)piperidin-3-yl](1-methyl-1H-imidazol-2-yl)methanone;
[1-(6,7-dimethoxyphthalazin-1-yl)piperidin-4-yl](phenyl)methanol;
[1-(6,7-dimethoxyphthalazin-1-yl)piperidin-4-yl](pyridin-2-yl)methanol;
[1-(6,7-dimethoxyphthalazin-1-yl)piperidin-4-yl](pyridin-3-yl)methanol;
[1-(6,7-dimethoxyphthalazin-1-yl)piperidin-4-yl](1-methyl-1H-imidazol-2-yl)methanol;
6,7-dimethoxy-1-[3-(5-methyl-1,2,4-oxadiazol-3-yl)piperidin-1-yl]phthalazine;
1′-(6,7-dimethoxyphthalazin-1-yl)-34-dihydro-2H-spiro[isoquinoline-14′-piperidine]
3-[1-(6,7-dimethoxyphthalazin-1-yl)piperidin-4-yl]phenol;
1-[3-(3-tert-butyl-1,2,4-oxadiazol-5-yl)piperidin-1-yl]-6,7-dimethoxyphthalazine;
1-[3-(4-chlorophenyl)piperidin-1-yl]-6,7-dimethoxyphthalazine;
6,7-dimethoxy-1-(4-pyridin-4-ylpiperidin-1-yl)phthalazine;
1-{3-[(2-fluorophenoxy)methyl]piperidin-1-yl}-6,7-dimethoxyphthalazine;
[1-(6,7-dimethoxyphthalazin-1-yl)piperidin-3-yl](phenyl)methanone;
[1-(6,7-dimethoxyphthalazin-1-yl)piperidin-3-yl](pyridin-2-yl)methanone;
6,7-dimethoxy-1-[3-(5-methyl-1,3,4-oxadiazol-2-yl)piperidin-1-yl]phthalazine;
6,7-dimethoxy-1-[4-(1H-pyrazol-5-yl)piperidin-1-yl]phthalazine;
[4-benzyl-1-(6,7-dimethoxyphthalazin-1-yl)piperidin-4-yl]methanol;
6,7-dimethoxy-1-[4-(2-methylphenyl)piperidin-1-yl]phthalazine;
1-(6,7-dimethoxyphthalazin-1-yl)-4-(4-methylphenyl)piperidin-4-ol;
6,7-dimethoxy-1-[3-(phenoxymethyl)piperidin-1-yl]phthalazine;
6,7-dimethoxy-1-(3-{[(2-methylpyridin-3-yl)oxy]methyl}piperidin-1-yl)phthalazine;
6,7-dimethoxy-1-(3-{[(6-methylpyridin-3-yl)oxy]methyl}piperidin-1-yl)phthalazine;
3-{[1-(6,7-dimethoxyphthalazin-1-yl)piperidin-3-yl]methoxy}pyridin-2-amine
1-(6,7-dimethoxyphthalazin-1-yl)-3-[(2-methyl-1H-imidazol-1-yl)methyl]piperidin-3-ol;
6,7-dimethoxy-1-{4-[5-(methoxymethyl)-1,3,4-oxadiazol-2-yl]piperidin-1-yl}phthalazine;
1-[4-(5-isobutyl-1,3,4-oxadiazol-2-yl)piperidin-1-yl]-6,7-dimethoxyphthalazine;
1-[3-(5-cyclopropyl-1,3,4-oxadiazol-2-yl)piperidin-1-yl]-6,7-dimethoxyphthalazine;
6,7-dimethoxy-1-{3-[5-(methoxymethyl)-1,3,4-oxadiazol-2-yl]piperidin-1-yl}phthalazine;
1-{5-[1-(6,7-dimethoxyphthalazin-1-yl)piperidin-3-yl]-1,3,4-oxadiazol-2-yl}-N,N-dimethylmethanamine
6,7-dimethoxy-1-{4-[(6-methylpyridazin-3-yl)methyl]piperidin-1-yl}phthalazine;
6,7-dimethoxy-1-[4-(pyrimidin-2-ylmethyl)piperidin-1-yl]phthalazine;
1-(6,7-dimethoxyphthalazin-1-yl)-3-(1H-pyrazol-1-ylmethyl)piperidin-3-ol;
6,7-dimethoxy-1-[4-(2-methylpyrimidin-4-yl)piperidin-1-yl]phthalazine;
6,7-dimethoxy-1-[3-(pyridin-2-ylmethyl)piperidin-1-yl]phthalazine;
6,7-dimethoxy-1-[3-(pyrimidin-2-ylmethyl)piperidin-1-yl]phthalazine;
(3-{[1-(6,7-dimethoxyphthalazin-1-yl)piperidin-3-yl]methyl}phenyl)methanol;
6,7-dimethoxy-1-{3-[2-(methoxymethyl)pyrimidin-4-yl]piperidin-1-yl}phthalazine;
6,7-dimethoxy-1-(3-pyrimidin-4-ylpiperidin-1-yl)phthalazine;
4-[1-(6,7-dimethoxyphthalazin-1-yl)piperidin-4-yl]-N-ethylpyrimidin-2-amine
6,7-dimethoxy-1-(4-pyrimidin-4-ylpiperidin-1-yl)phthalazine;
1-(6,7-dimethoxyphthalazin-1-yl)-4-[(2-methyl-1H-imidazol-1-yl)methyl]piperidin-4-ol;
6,7-dimethoxy-1-(4-[1,2,4]triazolo[15-a]pyrimidin-7-ylpiperidin-1-yl)phthalazine;
1-[4-(2-cyclopropylpyrimidin-4-yl)piperidin-1-yl]-6,7-dimethoxyphthalazine;
1-[4-(5-cyclopropyl-1,3,4-oxadiazol-2-yl)piperidin-1-yl]-6,7-dimethoxyphthalazine;
6,7-dimethoxy-1-[4-(pyrazin-2-ylmethyl)piperidin-1-yl]phthalazine;
3-[1-(6,7-dimethoxyphthalazin-1-yl)piperidin-4-yl]benzamide
4-[1-(6,7-dimethoxyphthalazin-1-yl)piperidin-4-yl]benzamide
6,7-dimethoxy-1-(4-pyrimidin-2-ylpiperidin-1-yl)phthalazine;
1-[3-(3-chlorobenzyl)piperidin-1-yl]-6,7-dimethoxyphthalazine;
6,7-dimethoxy-1-[3-(pyrimidin-5-ylmethyl)piperidin-1-yl]phthalazine;
4-[1-(6,7-dimethoxyphthalazin-1-yl)piperidin-4-yl]-N,N-dimethylpyrimidin-2-amine
6,7-dimethoxy-1-[4-(pyrimidin-5-ylmethyl)piperidin-1-yl]phthalazine;
1-[3-(1H-benzimidazol-2-yl)piperidin-1-yl]-6,7-dimethoxyphthalazine;
1-[4-(4-fluorobenzyl)piperidin-1-yl]-6,7-dimethoxyphthalazine;
6,7-dimethoxy-1-[3-(3-methylphenyl)piperidin-1-yl]phthalazine;
1-[4-(2-fluorobenzyl)piperidin-1-yl]-6,7-dimethoxyphthalazine;
6,7-dimethoxy-1-{3-[(2-methyl-1H-imidazol-1-yl)methyl]piperidin-1-yl}phthalazine;
6,7-dimethoxy-1-[4-(1H-pyrazol-1-ylmethyl)piperidin-1-yl]phthalazine;
1-[4-(3-cyclopropyl-1,2,4-oxadiazol-5-yl)piperidin-1-yl]-6,7-dimethoxyphthalazine;
1-[3-(5-cyclopropyl-4H-1,2,4-triazol-3-yl)piperidin-1-yl]-6,7-dimethoxyphthalazine;
1-(6,7-dimethoxyphthalazin-1-yl)-4-(2-fluoro-5-methylphenyl)piperidin-4-ol;
1-[4-(2-fluorophenoxy)piperidin-1-yl]-6,7-dimethoxyphthalazine;
1-{3-[(4-fluorophenoxy)methyl]piperidin-1-yl}-6,7-dimethoxyphthalazine;
6,7-dimethoxy-1-[4-(pyridin-2-ylmethoxy)piperidin-1-yl]phthalazine;
6,7-dimethoxy-1-{4-[5-(methoxymethyl)-1,2,4-oxadiazol-3-yl]piperidin-1-yl}phthalazine;
6,7-dimethoxy-1-[4-(3-methoxyphenyl)piperidin-1-yl]phthalazine;
1-[4-(3,5-difluorophenyl)piperidin-1-yl]-6,7-dimethoxyphthalazine;
1-[4-(3-fluorophenoxy)piperidin-1-yl]-6,7-dimethoxyphthalazine;
1-[4-(4-fluorophenoxy)piperidin-1-yl]-6,7-dimethoxyphthalazine;
4-[1-(6,7-dimethoxyphthalazin-1-yl)piperidin-4-yl]benzonitrile
1′-(6,7-dimethoxyphthalazin-1-yl)spiro[chromene-2,4′-piperidine]
1-[3-(1H-imidazol-2-yl)piperidin-1-yl]-6,7-dimethoxyphthalazine;
1′-(6,7-dimethoxyphthalazin-1-yl)-34-dihydrospiro[isochromene-1,4′-piperidine]
N-[1-(6,7-dimethoxyphthalazin-1-yl)piperidin-4-yl]pyrimidin-2-amine
N-[1-(6,7-dimethoxyphthalazin-1-yl)piperidin-4-yl]pyridin-2-amine
N-benzyl-1-(6,7-dimethoxyphthalazin-1-yl)-N-methylpiperidin-4-amine
6,7-dimethoxy-1-[4-(4-methylbenzyl)piperidin-1-yl]phthalazine;
1-[4-(4-chlorophenyl)-4-methylpiperidin-1-yl]-6,7-dimethoxyphthalazine;
1′-(6,7-dimethoxyphthalazin-1-yl)spiro[indole-34′-piperidin]-2(1H)-one;
4-phenyl-1-(5,6,7-trimethoxyphthalazin-1-yl)piperidin-4-ol;
4-benzyl-1-(5,6,7-trimethoxyphthalazin-1-yl)piperidin-4-ol;
phenyl[1-(5,6,7-trimethoxyphthalazin-1-yl)piperidin-4-yl]methanone;
1-[4-(1H-123-benzotriazol-1-yl)piperidin-1-yl]-5,6,7-trimethoxyphthalazine;
5,6,7-trimethoxy-1-[4-(3-methylphenoxy)piperidin-1-yl]phthalazine;
5,6,7-trimethoxy-1-[4-(2-methyl phenoxy)piperidin-1-yl]phthalazine;
5,6,7-trimethoxy-1-(4-pyridin-2-ylpiperidin-1-yl)phthalazine;
1-(4-benzylpiperidin-1-yl)-5,6,7-trimethoxyphthalazine;
1-[4-(benzyloxy)piperidin-1-yl]-5,6,7-trimethoxyphthalazine;
4-phenyl-1-(5,6,7-trimethoxyphthalazin-1-yl)piperidine-4-carbonitrile
4-(3-fluorophenyl)-1-(5,6,7-trimethoxyphthalazin-1-yl)piperidin-4-ol;
5,6,7-trimethoxy-1-(4-phenoxypiperidin-1-yl)phthalazine;
5,6,7-trimethoxy-1-[4-(2-methoxyphenoxy)piperidin-1-yl]phthalazine;
5,6,7-trimethoxy-1-(3-phenylpiperidin-1-yl)phthalazine;
5,6,7-trimethoxy-1-(3-phenoxypiperidin-1-yl)phthalazine;
5,6,7-trimethoxy-1-[3-(2-methoxyphenyl)piperidin-1-yl]phthalazine;
1-[4-(3-ethyl-1,2,4-oxadiazol-5-yl)piperidin-1-yl]-5,6,7-trimethoxyphthalazine;
1-[3-(3-ethyl-1,2,4-oxadiazol-5-yl)piperidin-1-yl]-5,6,7-trimethoxyphthalazine;
2-{[1-(5,6,7-trimethoxyphthalazin-1-yl)piperidin-4-yl]oxy}benzonitrile
1-[4-(5-ethyl-1,2,4-oxadiazol-3-yl)piperidin-1-yl]-5,6,7-trimethoxyphthalazine;
5,6,7-trimethoxy-1-[4-(3-methoxyphenoxy)piperidin-1-yl]phthalazine;
5,6,7-trimethoxy-1-[4-(4-methoxyphenoxy)piperidin-1-yl]phthalazine;
1-[4-(2-fluorophenyl)piperidin-1-yl]-5,6,7-trimethoxyphthalazine;
5,6,7-trimethoxy-1-[3-(3-methyl-1,2,4-oxadiazol-5-yl)piperidin-1-yl]phthalazine;
5,6,7-trimethoxy-1-[3-(4-methylphenoxy)piperidin-1-yl]phthalazine;
1-[4-(3,5-dimethyl-4H-1,2,4-triazol-4-yl)piperidin-1-yl]-5,6,7-trimethoxyphthalazine;
5,6,7-trimethoxy-1-[3-(3-methylphenoxy)piperidin-1-yl]phthalazine;
5,6,7-trimethoxy-1-[3-(2-methoxyphenoxy)piperidin-1-yl]phthalazine;
5,6,7-trimethoxy-1-[3-(4-methoxyphenoxy)piperidin-1-yl]phthalazine;
5,6,7-trimethoxy-1-[4-(3-methyl-1,2,4-oxadiazol-5-yl)piperidin-1-yl]phthalazine;
5,6,7-trimethoxy-1-[3-(4-methyl-4H-1,2,4-triazol-3-yl)piperidin-1-yl]phthalazine;
4-(4-fluorophenyl)-1-(5,6,7-trimethoxyphthalazin-1-yl)piperidin-4-ol;
5,6,7-trimethoxy-1-[3-(3-methoxyphenyl)piperidin-1-yl]phthalazine;
3-(24-dimethylphenyl)-1-(5,6,7-trimethoxyphthalazin-1-yl)piperidin-3-ol;
3-(2-ethylphenyl)-1-(5,6,7-trimethoxyphthalazin-1-yl)piperidin-3-ol;
1-[4-phenyl-1-(5,6,7-trimethoxyphthalazin-1-yl)piperidin-4-yl]ethanone;
1-[4-(13-benzoxazol-2-yl)piperidin-1-yl]-5,6,7-trimethoxyphthalazine;
1-[3-(benzyloxy)piperidin-1-yl]-5,6,7-trimethoxyphthalazine;
1-[3-(13-benzoxazol-2-yl)piperidin-1-yl]-5,6,7-trimethoxyphthalazine;
pyridin-3-yl[1-(5,6,7-trimethoxyphthalazin-1-yl)piperidin-3-yl]methanone;
(1-methyl-1H-imidazol-2-yl)[1-(5,6,7-trimethoxyphthalazin-1-yl)piperidin-3-yl]methanone;
phenyl[1-(5,6,7-trimethoxyphthalazin-1-yl)piperidin-4-yl]methanol;
pyridin-2-yl[1-(5,6,7-trimethoxyphthalazin-1-yl)piperidin-4-yl]methanol;
pyridin-3-yl[1-(5,6,7-trimethoxyphthalazin-1-yl)piperidin-4-yl]methanol;
(1-methyl-1H-imidazol-2-yl)[1-(5,6,7-trimethoxyphthalazin-1-yl)piperidin-4-yl]methanol;
5,6,7-trimethoxy-1-[3-(5-methyl-1,2,4-oxadiazol-3-yl)piperidin-1-yl]phthalazine;
1′-(5,6,7-trimethoxyphthalazin-1-yl)-34-dihydro-2H-spiro[isoquinoline-14′-piperidine];
3-[1-(5,6,7-trimethoxyphthalazin-1-yl)piperidin-4-yl]phenol;
1-[3-(3-tert-butyl-1,2,4-oxadiazol-5-yl)piperidin-1-yl]-5,6,7-trimethoxyphthalazine;
1-[3-(4-chlorophenyl)piperidin-1-yl]-5,6,7-trimethoxyphthalazine;
5,6,7-trimethoxy-1-(4-pyridin-4-ylpiperidin-1-yl)phthalazine;
1-{3-[(2-fluorophenoxy)methyl]piperidin-1-yl}-5,6,7-trimethoxyphthalazine;
phenyl[1-(5,6,7-trimethoxyphthalazin-1-yl)piperidin-3-yl]methanone;
pyridin-2-yl[1-(5,6,7-trimethoxyphthalazin-1-yl)piperidin-3-yl]methanone;
5,6,7-trimethoxy-1-[3-(5-methyl-1,3,4-oxadiazol-2-yl)piperidin-1-yl]phthalazine;
5,6,7-trimethoxy-1-[4-(1H-pyrazol-5-yl)piperidin-1-yl]phthalazine;
[4-benzyl-1-(5,6,7-trimethoxyphthalazin-1-yl)piperidin-4-yl]methanol;
5,6,7-trimethoxy-1-[4-(2-methylphenyl)piperidin-1-yl]phthalazine;
4-(4-methylphenyl)-1-(5,6,7-trimethoxyphthalazin-1-yl)piperidin-4-ol;
5,6,7-trimethoxy-1-[3-(phenoxymethyl)piperidin-1-yl]phthalazine;
5,6,7-trimethoxy-1-(3-{[(2-methylpyridin-3-yl)oxy]methyl}piperidin-1-yl)phthalazine;
5,6,7-trimethoxy-1-(3-{[(6-methylpyridin-3-yl)oxy]methyl}piperidin-1-yl)phthalazine;
3-{[1-(5,6,7-trimethoxyphthalazin-1-yl)piperidin-3-yl]methoxy}pyridin-2-amine;
3-[(2-methyl-1H-imidazol-1-yl)methyl]-1-(5,6,7-trimethoxyphthalazin-1-yl)piperidin-3-ol;
5,6,7-trimethoxy-1-{-4-[5-(methoxymethyl)-1,3,4-oxadiazol-2-yl]piperidin-1-yl}phthalazine;
1-[4-(5-isobutyl-1,3,4-oxadiazol-2-yl)piperidin-1-yl]-5,6,7-trimethoxyphthalazine;
1-[3-(5-cyclopropyl-1,3,4-oxadiazol-2-yl)piperidin-1-yl]-5,6,7-trimethoxyphthalazine;
5,6,7-trimethoxy-1-{3-[5-(methoxymethyl)-1,3,4-oxadiazol-2-yl]piperidin-1-yl}phthalazine;
N,N-dimethyl-1-{5-[1-(5,6,7-trimethoxyphthalazin-1-yl)piperidin-3-yl]-1,3,4-oxadiazol-2-yl}methanamine
5,6,7-trimethoxy-1-{4-[(6-methylpyridazin-3-yl)methyl]piperidin-1-yl}phthalazine;
5,6,7-trimethoxy-1-[4-(pyrimidin-2-ylmethyl)piperidin-1-yl]phthalazine;
3-(1H-pyrazol-1-ylmethyl)-1-(5,6,7-trimethoxyphthalazin-1-yl)piperidin-3-ol;
5,6,7-trimethoxy-1-[4-(2-methylpyrimidin-4-yl)piperidin-1-yl]phthalazine;
5,6,7-trimethoxy-1-[3-(pyridin-2-ylmethyl)piperidin-1-yl]phthalazine;
5,6,7-trimethoxy-1-[3-(pyrimidin-2-ylmethyl)piperidin-1-yl]phthalazine;
(3-{[1-(5,6,7-trimethoxyphthalazin-1-yl)piperidin-3-yl]methyl}phenyl)methanol;
5,6,7-trimethoxy-1-{3-[2-(methoxymethyl)pyrimidin-4-yl]piperidin-1-yl}phthalazine;
5,6,7-trimethoxy-1-(3-pyrimidin-4-ylpiperidin-1-yl)phthalazine;
N-ethyl-4-[1-(5,6,7-trimethoxyphthalazin-1-yl)piperidin-4-yl]pyrimidin-2-amine
5,6,7-trimethoxy-1-(4-pyrimidin-4-ylpiperidin-1-yl)phthalazine;
4-[(2-methyl-1H-imidazol-1-yl)methyl]-1-(5,6,7-trimethoxyphthalazin-1-yl)piperidin-4-ol;
5,6,7-trimethoxy-1-(4-[1,2,4]triazolo[15-a]pyrimidin-7-ylpiperidin-1-yl)phthalazine;
1-[4-(2-cyclopropylpyrimidin-4-yl)piperidin-1-yl]-5,6,7-trimethoxyphthalazine;
1-[4-(5-cyclopropyl-1,3,4-oxadiazol-2-yl)piperidin-1-yl]-5,6,7-trimethoxyphthalazine;
5,6,7-trimethoxy-1-[4-(pyrazin-2-ylmethyl)piperidin-1-yl]phthalazine;
3-[1-(5,6,7-trimethoxyphthalazin-1-yl)piperidin-4-yl]benzamide;
4-[1-(5,6,7-trimethoxyphthalazin-1-yl)piperidin-4-yl]benzamide;
5,6,7-trimethoxy-1-(4-pyrimidin-2-ylpiperidin-1-yl)phthalazine;
1-[3-(3-chlorobenzyl)piperidin-1-yl]-5,6,7-trimethoxyphthalazine;
5,6,7-trimethoxy-1-[3-(pyrimidin-5-ylmethyl)piperidin-1-yl]phthalazine;
N,N-dimethyl-4-[1-(5,6,7-trimethoxyphthalazin-1-yl)piperidin-4-yl]pyrimidin-2-amine
5,6,7-trimethoxy-1-[4-(pyrimidin-5-ylmethyl)piperidin-1-yl]phthalazine;
1-[3-(1H-benzimidazol-2-yl)piperidin-1-yl]-5,6,7-trimethoxyphthalazine;
1-[4-(4-fluorobenzyl)piperidin-1-yl]-5,6,7-trimethoxyphthalazine;
5,6,7-trimethoxy-1-[3-(3-methylphenyl)piperidin-1-yl]phthalazine;
1-[4-(2-fluorobenzyl)piperidin-1-yl]-5,6,7-trimethoxyphthalazine;
5,6,7-trimethoxy-1-{3-[(2-methyl-1H-imidazol-1-yl)methyl]piperidin-1-yl}phthalazine;
5,6,7-trimethoxy-1-[4-(1H-pyrazol-1-ylmethyl)piperidin-1-yl]phthalazine;
1-[4-(3-cyclopropyl-1,2,4-oxadiazol-5-yl)piperidin-1-yl]-5,6,7-trimethoxyphthalazine;
1-[3-(5-cyclopropyl-4H-1,2,4-triazol-3-yl)piperidin-1-yl]-5,6,7-trimethoxyphthalazine;
4-(2-fluoro-5-methylphenyl)-1-(5,6,7-trimethoxyphthalazin-1-yl)piperidin-4-ol;
1-[4-(2-fluorophenoxy)piperidin-1-yl]-5,6,7-trimethoxyphthalazine;
1-{3-[(4-fluorophenoxy)methyl]piperidin-1-yl}-5,6,7-trimethoxyphthalazine;
5,6,7-trimethoxy-1-[4-(pyridin-2-ylmethoxy)piperidin-1-yl]phthalazine;
5,6,7-trimethoxy-1-{-4-[5-(methoxymethyl)-1,2,4-oxadiazol-3-yl]piperidin-1-yl}phthalazine;
5,6,7-trimethoxy-1-[4-(3-methoxyphenyl)piperidin-1-yl]phthalazine;
1-[4-(3,5-difluorophenyl)piperidin-1-yl]-5,6,7-trimethoxyphthalazine;
1-[4-(3-fluorophenoxy)piperidin-1-yl]-5,6,7-trimethoxyphthalazine;
1-[4-(4-fluorophenoxy)piperidin-1-yl]-5,6,7-trimethoxyphthalazine;
4-[1-(5,6,7-trimethoxyphthalazin-1-yl)piperidin-4-yl]benzonitrile
1′-(5,6,7-trimethoxyphthalazin-1-yl)spiro[chromene-24′-piperidine]
1-[3-(1H-imidazol-2-yl)piperidin-1-yl]-5,6,7-trimethoxyphthalazine;
1′-(5,6,7-trimethoxyphthalazin-1-yl)-34-dihydrospiro[isochromene-14′-piperidine]
N-[1-(5,6,7-trimethoxyphthalazin-1-yl)piperidin-4-yl]pyrimidin-2-amine
N-[1-(5,6,7-trimethoxyphthalazin-1-yl)piperidin-4-yl]pyridin-2-amine
N-benzyl-N-methyl-1-(5,6,7-trimethoxyphthalazin-1-yl)piperidin-4-amine
5,6,7-trimethoxy-1-[4-(4-methyl benzyl)piperidin-1-yl]phthalazine;
1-[4-(4-chlorophenyl)-4-methylpiperidin-1-yl]-5,6,7-trimethoxyphthalazine;
1′-(5,6,7-trimethoxyphthalazin-1-yl)spiro[indole-34′-piperidin]-2(1H)-one;
and pharmaceutical salts thereof.

12. A pharmaceutical composition for treating psychotic disorders, delusional disorders and drug induced psychosis; anxiety disorders, movement disorders, mood disorders, neurodegenerative disorders and drug addiction, comprising an amount of a compound of formula I according to claim 1 effective in treating said disorder or condition.

13. A method of treating a disorder selected from psychotic disorders, delusional disorders and drug induced psychosis; anxiety disorders, movement disorders, mood disorders, and neurodegenerative disorders, which method comprises administering an amount of a compound of formula I according to claim 1 effective in treating said disorder.

14. The method of claim 13, wherein said disorder are selected from the group consisting of: dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; age-related cognitive decline, major depressive episode of the mild, moderate or severe type; a manic or mixed mood episode; a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder comprising a delusional disorder or schizophrenia; a bipolar disorder comprising bipolar I disorder, bipolar II disorder, cyclothymic disorder, Parkinson's disease; Huntington's disease; dementia, Alzheimer's disease, multi-infarct dementia, AIDS-related dementia, Fronto temperal Dementia; neurodegeneration associated with cerebral trauma; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; multi-system atrophy, paranoid, disorganized, catatonic, undifferentiated or residual type; schizophreniform disorder; schizoaffective disorder of the delusional type or the depressive type; delusional disorder; substance-induced psychotic disorder, psychosis induced by alcohol, amphetamine, cannabis, cocaine, hallucinogens, inhalants, opioids, or phencyclidine; personality disorder of the paranoid type; and personality disorder of the schizoid type.
